(19) United States
(12) Patent Application Publication
(10) Pub. No.: US 2008/0305040 A1
(43) Pub. Date: Dec. 11, 2008

(54) In Vivo or in Vitro Method For Detecting Amyloid Deposits Having at Least One Amyloidogenic Protein

(75) Inventor: William E. Klunk, Pittsburgh, PA (US)

Correspondence Address: FOLEY AND LARDNER LLP;SUITE 500, 3000 K STREET NW, WASHINGTON, DC, 20007, US

(73) Assignee: UNIVERSITY OF PITTSBURGH

(21) Appl. No.: 11/997,371

(22) PCT Filed: Sep. 14, 2006
(86) PCT No.: PCT/US2006/035823

§ 371 Date: Jun. 3, 2008

Related U.S. Application Data
(60) Provisional application No. 60/717,242, filed on Sep. 16, 2005.

(51) Int. Cl. A61K 51/00; A61K 49/00; C12Q 1/02; A61K 49/06

(52) U.S. Cl. 424/1.65; 424/9.1; 435/29; 424/9.34

(57) ABSTRACT

An amyloid deposit can be detected by administering to a subject or applying to a sample a compound of Formula (I) or Formula (II) or structures 1-45, as described, and then imaging to detect binding of the compound to an amyloid deposit, where the amyloido genie protein of the deposit can be AL, AH, ATTR, Aβ2M, AA, AApoAI, AApoAII, AGeI, ALys, AFib, ACys, ABri, ADan, APrP, ACaI, AlAPP, AANF, APro, AIns, AMed, AKer, A(tbn), and/or ALac.

### BACKGROUND

Amyloidosis is a slowly progressive condition, which can lead to significant morbidity and death. A diverse group of disease processes fall under the “amyloidosis” rubric, which is characterized by extracellular tissue deposits, in one or many organs, of various insoluble fibrillar proteins, generically termed “amyloid,” in amounts sufficient to impair normal function.

Amyloid deposits are extracellular and not metabolized or cleared by the body. Amyloid may be distinguished grossly by a starch-like staining reaction with iodine; hence the name amyloid. Microscopically, amyloid is differentiated by its extracellular distribution, by its tinctorial and optical properties when stained with Congo red, and by its protein fibril structure. Thus, under light microscopy, amyloid is a homogeneous, highly refractile substance with an affinity for Congo red dye, both in fixed tissues and in vivo. Under electron microscopy, amyloid consists of 100 Å (10 nm), linear nonbranching fibrils; under x-ray diffraction, it has a cross-beta pattern.

The diseases associated with amyloidosis are all typified by an accumulation amyloid deposits. The amyloid deposits are characterized by the presence of one or more amyloidogenic proteins, which are derived from precursor proteins that either have an abnormal structure or are abnormally increased in the serum.

The cause of amyloid production and its deposition in tissues is unknown. In the different biochemical types of amyloidosis, etiologic mechanisms may vary. In secondary amyloidosis, for example, a defect in the metabolism of the precursor protein (the acute-phase reactant: serum amyloid A) may exist, whereas in hereditary amyloidosis a genetically variant protein appears to be present. In primary amyloidosis, a monoclonal population of marrow cells produces fragments of or whole light chains that may be processed abnormally to form amyloid.

Three major types of amyloid and several less common forms have been defined biochemically. The first type, which has an N-terminal sequence that is homologous to a portion of the variable region of an immunoglobulin light chain, is called AL and occurs in primary amyloidosis and in amyloidosis associated with multiple myeloma. The second type has a unique N-terminal sequence of a nonimmunoglobulin protein called AA protein and occurs in patients with secondary amyloidosis. The third type, which is associated with familial amyloid polyneuropathy, is usually a transthyretin (prealbumin) molecule that has a single amino acid substitution. Other hereditary amyloids have been found to consist of mutant gelsolin in some families, mutant apolipoprotein A-I in several others, and other mutant proteins in hereditary cerebral artery amyloid. In the amyloid associated with chronic hemodialysis, 2-microglobulin has constituted amyloid protein. Amyloid associated with aging in skin and with endocrine organs may represent other biochemical forms of amyloidosis. The amyloid found in the histopathologic lesions of Alzheimer's disease consists of proteins. Chemical analyses relating to various forms of amyloidosis have led to a more refined classification. A unique protein, a pentraxin called AP (or serum AP), is universally associated with all forms of amyloid and forms the basis of a diagnostic test.

Three major systemic clinical forms are recognized currently. Amyloidosis is classified as primary or idiopathic (AL form) when there is no associated disease, and secondary, acquired, or reactive (AA form) when associated with chronic diseases, either infectious (tuberculosis, bronchiectasis, osteomyelitis, leprosy) or inflammatory (rheumatoid arthritis, granulomatous ileitis). Amyloid also is associated with multiple myeloma (AL), Hodgkin's disease (AA), other tumors, and familial Mediterranean fever (AA). Amyloidosis may accompany aging. The third major type appears in familial forms unassociated with other disease, often with distinctive types of neuropathy, nephropathy, and cardiopathy.

In primary (AL) amyloidosis, the heart, lung, skin, tongue, thyroid gland, and intestinal tract may be involved. Localized amyloid “tumors” may be found in the respiratory tract or other sites. Parenchymal organs (liver, spleen, kidney) and the vascular system, especially the heart, are involved frequently.

Secondary (AA) amyloidosis shows a predilection for the spleen, liver, kidney, adrenals, and lymph nodes. No organ system is spared, however, and vascular involvement may be widespread, though clinically significant involvement of the heart is rare. The liver and spleen often are enlarged, firm, and rubbery. The kidneys usually are enlarged. Sections of the spleen have large, translucent, waxy areas where the normal malpighian bodies are replaced by pale amyloid, producing the sago spleen.

Hereditary amyloidosis is characterized by a peripheral sensory and motor neuropathy, often autonomic neuropathy, and cardiovascular and renal amyloid. Carpal tunnel syndrome and vitreous abnormalities may occur.

Amyloid associated with certain malignancies (e.g., multiple myeloma) has the same distribution as idiopathic (AL) amyloid; with other malignancies (e.g., medullary carcinoma of the thyroid gland) it may occur only locally in association with the tumor or in metastases. Amyloid frequently is found in the pancreas of individuals with adult-onset diabetes mellitus.

While amyloidosis may be suspected on the basis of specific clinical symptoms and signs, it can be definitively diagnosed only by biopsy. Currently, subcutaneous abdominal fat pad aspiration and biopsy of rectal mucosa are the best screening tests. Other useful biopsy sites are gingiva, skin, nerve, kidney, and liver. Tissue sections should be stained with Congo red dye and observed with a polarizing microscope for the characteristic green birefringence of amyloid. Isotopically labeled serum AP has been used in a scintigraphic test to confirm the diagnosis of amyloidosis. Better diagnostic methodologies need to be developed in order to provide early diagnosis thereby permitting effective treatment.

### SUMMARY OF THE INVENTION

The present invention relates to an in vivo or in vitro method for detecting in a subject at least one amyloid deposit comprising at least one amyloidogenic protein, comprising the steps of:

(a) administering to a subject suffering from a disease associated with amyloidosis, a detectable quantity of a pharmaceutical composition comprising at least one compound of formula I and a pharmaceutically acceptable carrier,

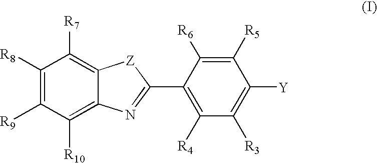

wherein

(i) Z is S, NR′, O or C(R′)₂, such that when Z is C(R′)₂, the tautomeric form of the heterocyclic ring may form an indole:

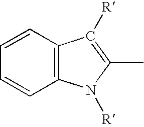

wherein R′ is H or a lower alkyl group,
(ii) Y is NR¹R², OR², or SR²,
(iii) R¹ is selected from the group consisting of H, a lower alkyl group, (CH₂)ₙOR′ (wherein n=1, 2, or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3, or 4 and Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR═, SR′ and COOR′, where R′ is H or a lower alkyl group);

(iv) R² is selected from the group consisting of H, a lower alkyl group, (CH₂)ₙOR′ (wherein n−1, 2, or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3, or 4 and Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′ and COOR′, where R′ is H or a lower alkyl group);

(v) R³ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;

(vi) R⁴ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;

(vii) R⁵ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ᵣOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;

(viii) R⁶ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;

(ix) R⁷ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;

(x) R⁸ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;

(xi) R⁹ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;

(xii) R¹⁰ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
alternatively, one of R³-R¹⁰ may be a chelating group, with or without a chelated metal group, said chelating group being of the form W-L or V-W-L, wherein V is selected from the group consisting of —COO—, —CO—, —CH₂O— and —CH₂NH—; W is —(CH₂)ₙ where n=0, 1, 2, 3, 4, or 5, and L is:

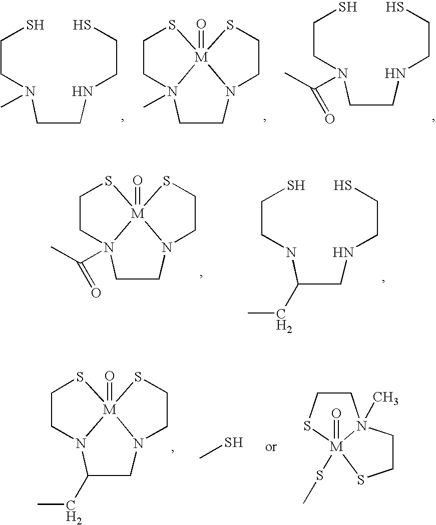

wherein M is selected from the group consisting of Tc and Re and radiolabelled derivatives and pharmaceutically acceptable salts thereof, where at least one of the substituent moieties comprises a detectable label; and

(b) detecting the binding of the compound to an amyloid deposit comprising at least one amyloidogenic protein, wherein the amyloidogenic protein is selected from the group consisting of AL, AH, ATTR, Aβ2M, AA, AApoAI, AApoAII, AGel, ALys, AFib, ACys, ABri, ADan, APrP, ACal, AlAPP, AANF, APro, AIns, AMed, AKer, A(tbn), and ALac.

The present invention additionally relates to an in vivo method for detecting at least one amyloid deposit, comprised of at least one amyloidogenic protein. The inventive method comprises the steps of:

(a) administering to a subject suffering from a disease associated with amyloidosis, a detectable quantity of a pharmaceutical composition comprising at least one compound of formula I, as defined above, and a pharmaceutically acceptable carrier,

whereby the compound binds to the amyloid deposit comprising at least one amyloidogenic protein, which is selected from the group consisting of AL, AH, ATTR, Aβ2M, AA, AApoAI, AApoAII, AGel, ALys, AFib, ACys, ABri, ADan, APrP, ACal, AlAPP, AANF, APro, AIns, AMed, AKer, A(tbn), and ALac;

(b) irradiating the subject and collecting imaging data emitted by the compound; and then

(c) processing the imaging data.

Additionally, the invention encompasses the use of a compound according to formula (I), as herein defined, for detecting at least one amyloid deposit in a subject suffering from a disease associated with amyloidosis. In a related vein, the invention further comprehends the use of a formula (I) compound in the preparation of a medicament for use in the detection of at least one amyloid deposit in such a subject.

In one embodiment, the amyloidogenic protein is derived from at least one protein precursor selected from the group consisting of immunoglobulin light chain, immunoglobulin heavy chain, transthyretin, P 2-microglobulin, (Apo)serum AA, Apolipoprotien AI, Apolipoprotein AII, gelsolin, lysozyme, fibrinogen α-chain, cystatin C, ABriPP, ADanPP, prion protein, (Pro)calcitonin, islet amyloid polypeptide, atrial natriuretic factor, prolactin, insulin, lactadherin, kerato-epithelin, Pindborg tumor associated precursor protein (tbn) and lactoferrin.

In one embodiment, the patient population encompasses a subject who is suffering from a disease associated with systemic amyloidosis.

In another embodiment, the patient population encompasses a subject who is suffering from cerebral amyloid angiopathy.

In another embodiment, the at least one amyloid deposit is located in a mesodermal tissue of the subject. In one aspect of this embodiment, the tissue is selected from the group consisting of peripheral nerve, skin, tongue, joint, heart or liver.

In further embodiment, an amyloid deposit is located in a parenchymatous organ. In one aspect of this embodiment, the organ is selected from the group consisting of spleen, kidney, liver, and adrenal.

In yet a further embodiment, the disease associated with systemic amyloidosis is selected from the group consisting of multiple myeloma, macroglobulinemia, lymphoma, chronic inflammatory disease, rheumatoird arthritis, infectious disease, dermatomyositis, scleroderma, regional enteritis, ulcerative colitis, tuberculosis, chronic osteomyelitis, bronchiectasis, skin abscess, lung abscess, cancer, Hodgkin's disease, heredofamilial amyloidosis, familial Mediterranean fever, familial dementia and familial amyloid polyneuropathy. In one aspect of this embodiment, the skin or lung abscess results from subcutaneous heroin use.

The inventive method comprehends detecting via an approach selected from the group consisting of gamma imaging, magnetic resonance imaging, and magnetic resonance spectroscopy. In an aspect of this embodiment, the detecting is done by gamma imaging, which is either PET or SPECT.

In still another embodiment, the pharmaceutical composition is administered by intravenous injection.

In a different embodiment, the patient population encompasses a subject who is receiving hemodialysis for chronic renal failure. In another embodiment, the subject is suffering from a disease associated with localized amyloidosis. In one aspect of this embodiment, the at least one amyloid deposit is located in a tissue selected from the group consisting of tenosynovium, joints, aortic, thyroid, islets of langerhans, aging pituitary, latrogenic, cardiac atria, and cornea. In one aspect of this embodiment, the at least one amyloid deposit is located in the pancreas. In one aspect of this embodiment, the disease associated with localized amyloidosis is selected from the group consisting of primary myeloma, familial dementia, spongioform encephalopathies, c-cell thyroid tumor, insulinoma, prolactinoma and pindborg tumor.

In some embodiments, the compound of Formula (I) comprises a compound of formula (II):

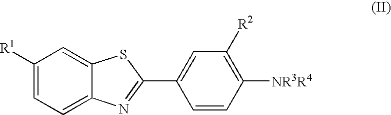

or a radiolabeled derivative, pharmaceutically acceptable salt, hydrate, solvate or prodrug of the compound, wherein:

R¹ is hydrogen, —OH, —NO₂, —CN, —COOR, —OCH₂OR, C₁-C₆ alkyl, C₂-C₆ alkenyl, C₂-C₆ alkynyl, C₁-C₆ alkoxy or halo;

R is C₁-C₆ alkyl;

R² is hydrogen or halo;

R³ is hydrogen, C₁-C₆ alkyl, C₂-C₆ alkenyl or C₂-C₆ alkynyl; and

R⁴ is hydrogen, C₁-C₆ alkyl, C₂-C₆ alkenyl or C₂-C₆ alkynyl, wherein the alkyl, alkenyl or alkynyl comprises a radioactive carbon or is substituted with a radioactive halo when R² is hydrogen or a non-radioactive halo;

provided that when R¹ is hydrogen or —OH, R² is hydrogen and R⁴ is —¹¹CH₃, then R³ is C₂-C₆ alkyl, C₂-C₆ alkenyl or C₂-C₆ alkynyl; and

further provided that when R¹ is hydrogen, R² hydrogen and R⁴ is —(CH₂)₃¹⁸F, then R³ is C₂-C₆ alkyl, C₂-C₆ alkenyl or C₂-C₆ alkynyl, where at least one of the substituent moieties comprises a detectable label.

In a still further embodiment, the amyloid imaging agent of formula (I) is selected from the group consisting of structures 1-45 or a radiolabeled derivative thereof, wherein the compound comprises at least one detectable label.

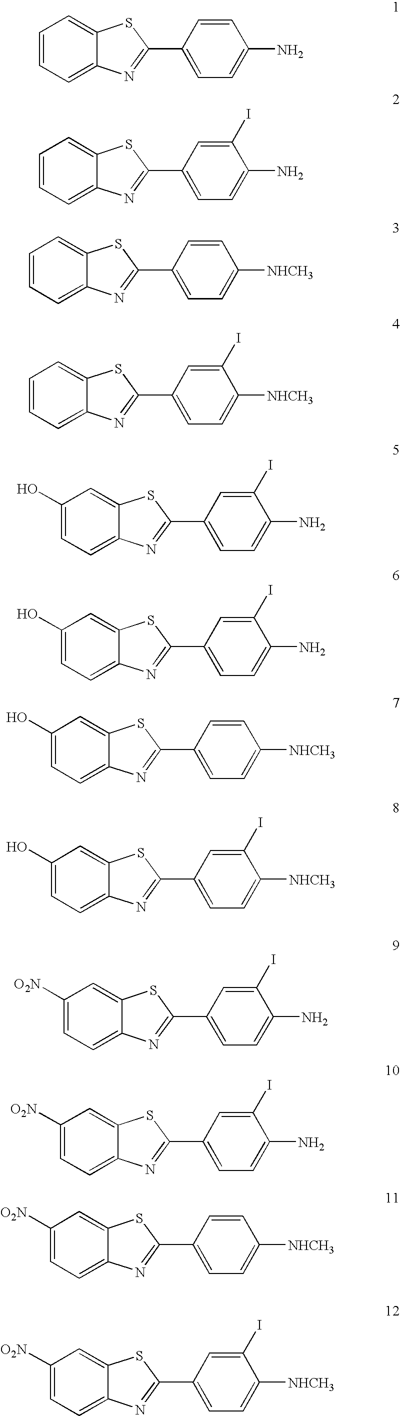

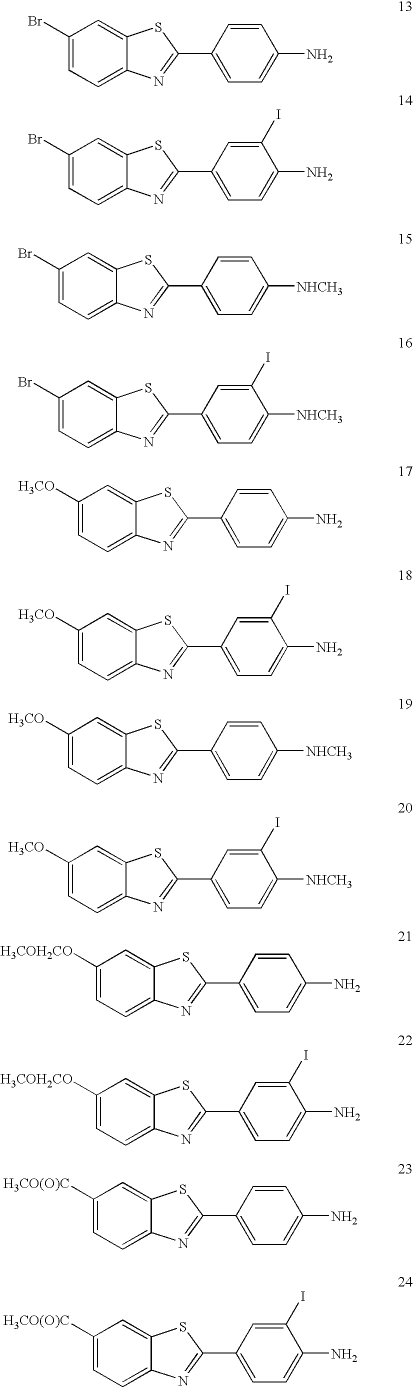

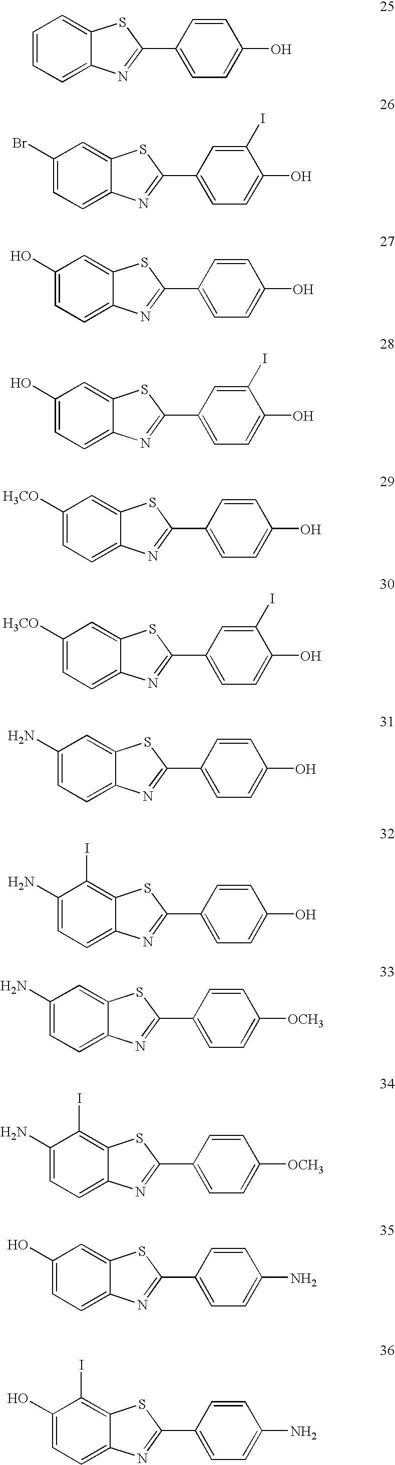

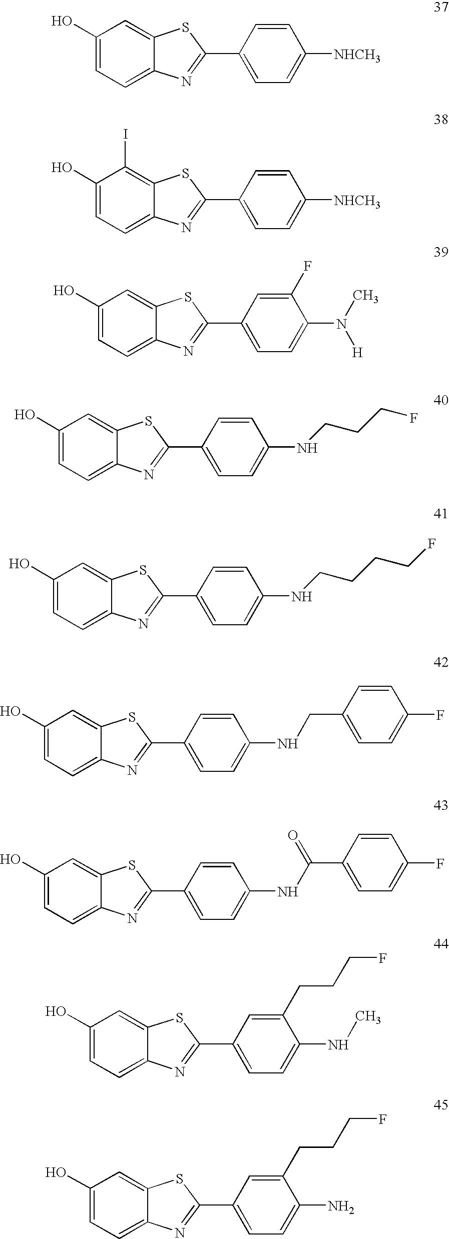

### BRIEF DESCRIPTION OF THE DRAWINGS

FIG. 1 shows binding X-34, a Chrysamine G derivative and 6-CN-BTA-1, a thioflavin derivative to amyloid deposits in heart tissue, lung tissue, bladder tissue, lymph node tissue and bone.

### DETAILED DESCRIPTION

As noted, “amyloidosis” connotes a pathological condition associated with amyloid deposition. Illustrative of such conditions are Alzheimer's Disease, Down's Syndrome, Type 2 diabetes mellitus, hereditary cerebral hemorrhage amyloidosis (Dutch), amyloid A (reactive), secondary amyloidosis, MCI, familial Mediterranean fever, familial amyloid nephropathy with urticaria and deafness (Muckle-wells Syndrome), amyloid lambda L-chain or amyloid kappa L-chain (idiopathic, myeloma or macroglobulinemia-associated) Aβ2M (chronic hemodialysis), ATTR (familial amyloid polyneuropathy (Portuguese, Japanese, Swedish)), familial amyloid cardiomyopathy (Danish), isolated cardiac amyloid, systemic senile amyloidoses, AIAPP or amylin insulinoma, atrial naturetic factor (isolated atrial amyloid), procalcitonin (medullary carcinoma of the thyroid), gelsolin (familial amyloidosis (Finnish)), cystatin C (hereditary cerebral hemorrhage with amyloidosis (Icelandic)), AApo-A-I (familial amyloidotic polyneuropathy-Iowa), AApo-A-II (accelerated senescence in mice), fibrinogen-associated amyloid, and Asor or Pr P-27 (scrapie, Creutzfeld Jacob disease, Gertsmann-Straussler-Scheinker syndrome, bovine spongiform encephalitis). Also included, are detection of amyloid diseases in persons who are homozygous for the apolipoprotein E4 allele, and in patients clinically diagnosed with Huntington's disease. The invention encompasses diseases associated with amyloid plaque deposition. The present invention is primarily focused on detecting amyloid deposits in non-cerebral tissues.

In accordance with the present invention, in vivo or in vitro detection is effected, in relation to a subject who has or who is at risk of having at least one amyloid deposit (i.e., a deposit comprised of at least one amyloidogenic protein), via a methodology that entails:

(a) administering to a subject suffering from a disease associated with amyloidosis, a detectable quantity of a pharmaceutical composition comprising at least one compound of formula:

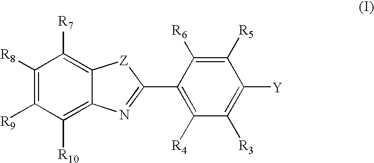

wherein

(i) Z is S, NR′, O or C(R′)₂, such that when Z is C(R′)₂, the tautomeric form of the heterocyclic ring may form an indole:

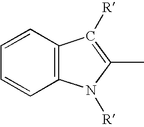

wherein R′ is H or a lower alkyl group,

(ii) Y is NR¹R², OR², or SR²,

(iii) R¹ is selected from the group consisting of H, a lower alkyl group,

(CH₂)ₙOR′ (wherein n=1, 2, or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3, or 4 and Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′ and COOR′, where R′ is H or a lower alkyl group);

(iv) R² is selected from the group consisting of H, a lower alkyl group, (CH₂)ₙOR′ (wherein n=1, 2, or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3, or 4 and Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′ and COOR′, where R′ is H or a lower alkyl group);

(v) R³ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;

(vi) R⁴ is selected from the group consisting of H, F, Cl, Br, I, a lower alkyl group, (CH₂)ₙOR′ (wherein n−1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;

(vii) R⁵ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;

(viii) R⁶ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;

(ix) R⁷ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;

(x) R⁸ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;

(xi) R⁹ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;

(xii) R¹⁰ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;

alternatively, one of R³-R¹⁰ may be a chelating group (with or without a chelated metal group) of the form W-L or V—W-L, wherein V is selected from the group consisting of —COO—, —CO—, —CH₂O— and —CH₂NH—; W is —(CH₂)ₙ where n=0, 1, 2, 3, 4, or 5; and L is:

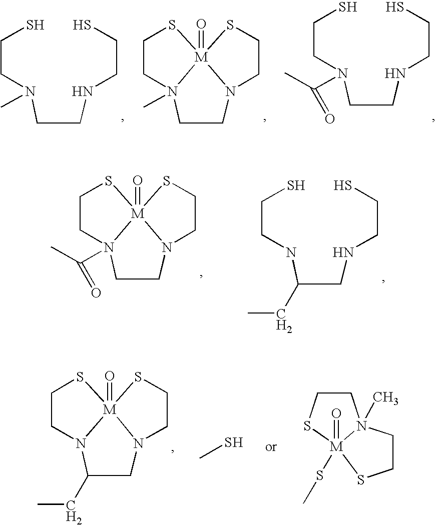

wherein M is selected from the group consisting of Tc and Re,

and radiolabeled derivatives and pharmaceutically acceptable salts thereof, where at least one of the substituent moieties comprises a detectable label; and

(b) detecting the binding of the compound to an amyloid deposit comprising at least one amyloidogenic protein, wherein the amyloidogenic protein is selected from the group consisting of AL, AH, ATTR, Aβ2M, AA, AApoAI, AApoAII, AGel, ALys, AFib, ACys, ABri, ADan, APrP, ACal, AlAPP, AANF, APro, AIns, AMed, AKer, A(tbn), and ALac.

In primary systemic amyloidosis (AL), the amyloidogenic protein is an abnormally conformed monoclonal immunoglobulin light chains (k or λ) produced by clonal plasma cells. Fibrils deposit in kidneys, heart, liver, and other organs/tissues.

In a few cases, immunoglobulin chain amyloidosis fibrils contain only heavy chain sequences rather than light chain sequences. In that circumstance, the disease is termed “heavy chain amyloidosis” (AH).

In transthyretin amyloidosis, the precursor protein is the normal or mutant sequence TTR, a transport protein synthesized in the liver and choroid plexus. TTR is a tetramer of 4 identical subunits of 127 amino acids each. Normal-sequence TTR forms amyloid deposits in the cardiac ventricles of elderly (>70 year-old) individuals; this disease is also called “senile cardiac amyloidosis.” The prevalence of TTR cardiac amyloidosis increases progressively with age, affecting 25% or more of the population older than 90 years. Normal-sequence ATTR can be an incidental autopsy finding, or it can cause clinical symptoms (e.g., heart failure and arrhythmias).

Point mutations in TTR increase the tendency of TTR to form amyloid. Amyloidogenic TTR mutations are inherited as an autosomal dominant disease with variable penetrance. More than 60 amyloidogenic TTR mutations are known. The most prevalent TTR mutations are TTR Val30Met (common in Portugal, Japan, and Sweden), and TTR Val122Ile (carried by 3.9% of African Americans). Amyloidogenic TTR mutations cause deposits primarily in the peripheral nerves, heart, gastrointestinal tract, and vitreous.

In β2-microglobulin amyloidosis, the precursor protein is a normal β-microglobulin (β2M), which is the light chain component of the major histocompatibility complex. In the clinical setting, Aβ2M is associated with patients on dialysis and, rarely, patients with renal failure who are not on dialysis.

β2M is normally catabolized in the kidney. In patients with renal failure, the protein accumulates in the serum. Conventional dialysis membranes do not remove β2M; therefore, serum levels can reach as high as 30-60 times the reference range values in patients on hemodialysis. Typical organs involved include the carpal ligament and, possibly, the synovial membranes (leading to arthropathies and bone cysts) and the heart, gastrointestinal tract, liver, lungs, prostate, adrenals, and tongue.

Amyloid A (AA) amyloidosis is the most common form of systemic amyloidosis worldwide. It occurs in the course of a chronic inflammatory disease of either infectious or noninfectious etiology. In AA, the kidney, liver, and spleen are the major sites of involvement.

Apolipoprotein AI amyloidosis (AApoAI) is an autosomal dominant amyloidosis caused by point mutations in the apoAI gene. Usually, this amyloidosis is a prominent renal amyloid. Some kindreds have peripheral neuropathy or cardiac disease. ApoAI (likely of normal sequence) also is the fibril precursor in localized amyloid plaques in the aortae of elderly people.

Apolipoprotein AII amyloidosis (AApoAII) is an autosomal dominant amyloidosis caused by point mutations in the apoAII gene. The 2 kindreds described with this disorder have each carried a point mutation in the stop codon, leading to production of an abnormally long protein.

The precursor protein in gelsolin amyloidosis (AGel) is the actin-modulating protein gelsolin. Amyloid fibrils include a gelsolin fragment that contains a point mutation.

Fibrinogen amyloidosis (AFib) is an autosomal dominant amyloidosis caused by point mutations in the fibrinogen alpha chain gene.

Lysozyme amyloidosis (ALys) is an autosomal dominant amyloidosis caused by point mutations in the lysozyme gene.

The precursor protein in cystatin C amyloidosis (ACys) is cystatin C, which is a cysteine protease inhibitor that contains a point mutation. This condition is clinically termed HCHWA, Icelandic type. ACys is autosomal dominant. Clinical presentation includes multiple strokes and mental status changes beginning in the second or third decade of life. The pathogenesis is one of mutant cystatin that is widely distributed in tissues, but fibrils form only in the cerebral vessels; therefore, local conditions are believed to play a role in fibril formation.

The precursor protein in prion protein amyloidosis (APrP) is a prion protein, which is a plasma membrane glycoprotein. The etiology is either infectious (i.e., kuru) or genetic (i.e., Creutzfeldt-Jakob disease (CJD), Gerstmann-Sträussler-Scheinker (GSS) syndrome, fatal familial insomnia (FFI)). The infectious unit is the prion protein, which induces a conformational change in a homologous protein encoded by a host chromosomal gene. Patients with CJD, GSS, and FFI carry autosomal dominant amyloidogenic mutations in the prion protein gene; therefore, the amyloidosis forms even in the absence of an infectious trigger.

In calcitonin amyloid (ACal), the precursor protein is calcitonin, a calcium regulatory hormone synthesized by the thyroid. Patients with medullary carcinoma of the thyroid may develop localized amyloid deposition in the tumors, consisting of normal-sequence procalcitonin (ACal). The presumed pathogenesis is increased local calcitonin production, leading to a sufficiently high local concentration of the peptide causing polymerization and fibril formation.

In islet amyloid polypeptide amyloidosis (AIAPP), the precursor protein is an islet amyloid polypeptide (IAPP), also known as amylin. IAPP is a protein secreted by the islet beta cells that are stored with insulin in the secretory granules and released in concert with insulin. Normally, IAPP modulates insulin activity in skeletal muscle. IAPP amyloid is found in insulinomas and in the pancreas of many patients with diabetes mellitus type 2.

Atrial natriuretic factor amyloidosis is associated with the precursor protein, atrial natriuretic factor (ANF), a hormone controlling salt and water homeostasis, which is synthesized by the cardiac atria. Amyloid deposits are localized to the cardiac atria. This condition is highly prevalent in elderly people. Atrial natriuretic factor amyloidosis (AANF) is most common in patients with long-standing congestive heart failure, presumably because of persistent ANF production.

In prolactin amyloid (APro), prolactin or prolactin fragments are found in the pituitary amyloid. This condition is often observed in elderly people and has also been reported in an amyloidoma in a patient with a prolactin-producing pituitary tumor.

Amyloids of the skin react with some antikeratin antibodies to generate a localized form of amyloidosis. However, the exact identity of the fibrils is not chemically confirmed in keratin amyloid, but they are referred to as keratin amyloid proteins (AKer).

Aortic medial amyloid occurs in most people older than 60 years. Medin amyloid (AMed) is derived from a proteolytic fragment of lactadherin, a glycoprotein expressed by mammary epithelium.

Familial British dementia (FBD) is characterized neuropathologically by deposition of a unique amyloid-forming protein, ABri. It is a fragment of an abnormal form of a precursor protein, BRI.

In Familial Danish dementia (FDD), a decamer duplication between codons 265 and 266 in the 3′ region of the BRI gene originates an amyloid peptide named ADan, 11 residues longer than the wild-type peptide produced from the normal BRI gene. ADan deposits have been found widely distributed in the CNS of FDD cases. The deposits of ADan are predominantly non-fibrillar aggregates.

The ABri and ADan peptides are fragments derived from a larger, membrane-anchored precursor protein, termed BRI precursor protein, and encoded by the BRI gene on chromosome 13.

Pindborg tumor is characterized by the production of large amounts of amyloid and the presence of calcified lamellar bodies. The amyloid protein associated with this syndrome has yet to be named but is commonly referred to as A(tbn).

Amyloid fibrils can be formed in the absence of serum amyloid P(SAP) component and heparin sulfate proteoglycans from several natural polypeptides, such as insulin. This gives rise to the amyloid protein, AIns, the precuror of which is insulin.

Another protein, lactoferrin, is reported as the major fibril protein in familial subepithelial corneal amyloidosis. It is presumed that either a structural abnormality or abnormally increased concentration in the serum gives rise to the amyloid protein ALac.

The amyloidogenic proteins are detected by the present thioflavin compounds. The thioflavin compounds target at least one amyloidogenic protein, which is derived from at least one protein precursor selected from the group consisting of immunoglobulin light chain, immunoglobulin heavy chain, transthyretin, β2-microglobulin, (Apo)serum AA, Apolipoprotien AI, Apolipoprotein AII, gelsolin, lysozyme, fibrinogen α-chain, cystatin C, ABriPP, ADanPP, prion protein, (Pro)calcitonin, islet amyloid polypeptide, atrial natriuretic factor, prolactin, insulin, lactadherin, kerato-epithelin, Pindborg tumor associated precursor protein (tbn) and lactoferrin. It is these protein targets that are believed to give rise to different syndromes or diseases of affected tissues. See Buxbaum, Curr. Opin Rheumatol 16: 67-75 (2003). See also, Merlini and Westermark, J Intern Med 255: 159-178 (2004).

The detectable label includes any atom or moiety which can be detected using an imaging technique known to those skilled in the art. Typically, the detectable label is selected from the group consisting of ³H, ¹³¹I, ¹²⁵I, ¹²³I, ⁷⁶Br, ⁷⁵Br, ¹⁸F, CH₂—CH₂—X*, O—CH₂—CH₂—X*, CH₂—CH₂—CH₂—X*, O—CH₂—CH₂—CH₂—X* (wherein X*=¹³¹I, ¹²³I, ⁷⁶Br, ⁷⁵Br or ¹⁸F), ¹⁹F, ¹²⁵I, a carbon-containing substituent selected from the group consisting of lower alkyl, (CH₂)ₙOR′, CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, (C═O)N(R′)₂, O(CO)R′, COOR′, CR′═CR′—Rₚₕ and CR₂′—CR₂′—Rₚₕ wherein at least one carbon is ¹¹C, ¹³C or ¹⁴C and a chelating group (with chelated metal group) of the form W-L* or V-W-L*, wherein V is selected from the group consisting of —COO—, —CO—, —CH₂O— and —CH₂NH—; W is —(CH₂)ₙ where n=0, 1, 2, 3, 4, or 5; and L* is:

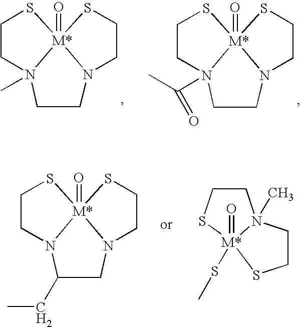

wherein M* is ^{99m}Tc.

In a preferred embodiment, the detectable label is a radiolabel.

Use of a compound of formula (I) in preparation of a medicament used in an in vivo method for detecting in a subject at least one amyloid deposit comprising at least one amyloidogenic protein,

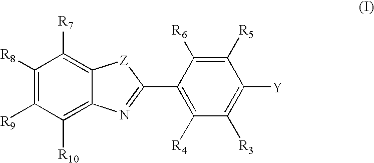

wherein

(i) Z is S, NR′, O or C(R′)₂, such that when Z is C(R′)₂, the tautomeric form of the heterocyclic ring may form an indole:

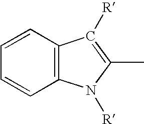

wherein R′ is H or a lower alkyl group,

(ii) Y is NR¹R², OR², or SR²,

(iii) R¹ is selected from the group consisting of H, a lower alkyl group, (CH₂)ₙOR′ (wherein n=1, 2, or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3, or 4 and Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′ and COOR′, where R′ is H or a lower alkyl group);

(iv) R² is selected from the group consisting of H, a lower alkyl group, (CH₂)ₙOR′ (wherein n=1, 2, or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3, or 4 and Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′ and COOR′, where R′ is H or a lower alkyl group);

(v) R³ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;

(vi) R⁴ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;

(vii) R⁵ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;

(viii) R⁶ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;

(ix) R⁷ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;

(x) R⁸ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;

(xi) R⁹ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;

(xii) R¹⁰ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
alternatively, one of R³-R¹⁰ may be a chelating group, with or without a chelated metal group, said chelating group being of the form W-L or V-W-L, wherein V is selected from the group consisting of —COO—, —CO—, —CH₂O— and —CH₂NH—; W is —(CH₂)ₙ where n=0, 1, 2, 3, 4, or 5, and L is:

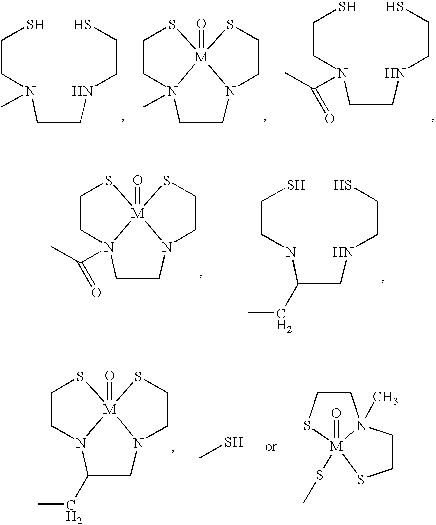

wherein M is selected from the group consisting of Tc and Re and radiolabelled derivatives and pharmaceutically acceptable salts thereof, where at least one of the substituent moieties comprises a detectable label,

comprising the steps of:

(a) administering to a subject suffering from a disease associated with amyloidosis, a detectable quantity of a pharmaceutical composition comprising at least one compound of formula I and a pharmaceutically acceptable carrier, and

(b) detecting the binding of the compound to an amyloid deposit comprising at least one amyloidogenic protein, wherein the amyloidogenic protein is selected from the group consisting of AL, AH, ATTR, Aβ2M, AA, AApoAI, AApoAII, AGel, ALys, AFib, ACys, ABri, ADan, APrP, ACal, AlAPP, AANF, APro, AIns, AMed, AKer, A(tbn), and ALac.

In one embodiment, the use of the compounds of Formula (I) referred to above are used in detection of proteins wherein the at least one amyloidogenic protein is derived from at least one protein precursor selected from the group consisting of immunoglobulin light chain, immunoglobulin heavy chain, transthyretin, β2-microglobulin, (Apo)serum AA, Apolipoprotien AI, Apolipoprotein AII, gelsolin, lysozyme, fibrinogen α-chain, cystatin C, ABriPP, ADanPP, prion protein, (Pro)calcitonin, islet amyloid polypeptide, atrial natriuretic factor, prolactin, insulin, lactadherin, kerato-epithelin, Pindborg tumor associated precursor protein (tbn) and lactoferrin.

In one embodiment, the use of the compounds of Formula (I) for preparation of a medicament for the in vivo method of detection of amyloidosis involves subjects suffering from a disease associated with systemic amyloidosis. In a preferred embodiment, the disease associated with systemic amyloidosis is selected from the group consisting of multiple myeloma, macroglobulinemia, lymphoma, chronic inflammatory disease, rheumatoird arthritis, infectious disease, dermatomyositis, scleroderma, regional enteritis, ulcerative colitis, tuberculosis, chronic osteomyelitis, bronchiectasis, skin abscess, lung abscess, cancer, Hodgkin's disease, heredofamilial amyloidosis, familial Mediterranean fever, familial dementia and familial amyloid polyneuropathy.

In one embodiment, the use of the compounds of Formula (I) for preparation of a medicament for the in vivo method of detection of amyloidosis involves detecting at least one amyloid deposit is located in a mesodermal tissue of the subject or a parenchymatous organ. In a preferred embodiment, the mesodermal tissue is selected from peripheral nerve, skin, tongue, joint, heart or liver. In a preferred embodiment, the organ is selected from the group consisting of spleen, kidney, liver and adrenal. In one embodiment, the skin or lung abscess results from subcutaneous heroin use.

In the embodiment involving the use of the compounds of Formula (I) for the preparation of a medicament for in vivo detection of amyloidosis the detecting is accomplished by a method selected from the group consisting of gamma imaging, magnetic resonance imaging and magnetic resonance spectroscopy. In a preferred embodiment, the gamma imaging is either PET or SPECT.

In the embodiment involving the use of the compounds of Formula (I) for the preparation of a medicament for in vivo detection of amyloidosis the medicament is administered by intravenous injection.

In one embodiment of involving the use of the compounds of Formula (I) for the preparation of a medicament for in vivo detection of amyloidosis, the subject is receiving hemodialysis for chronic renal failure.

In one embodiment involving the use of the compounds of Formula (I) for the preparation of a medicament for in vivo detection of amyloidosis, the subject is suffering from a disease associated with localized amyloidosis. In a preferred embodiment, the at least one amyloid deposit is located in a tissue selected from the group consisting of tenosynovium, joints, aortic, thyroid, islets of langerhans, aging pituitary, latrogenic, cardiac atria, and cornea. In one embodiment, the at least one amyloid deposit is located in the pancreas. In this embodiment, the disease associated with localized amyloidosis is selected from the group consisting of primary myeloma, familial dementia, spongioform encephalopathies, c-cell thyroid tumor, insulinoma, prolactinoma and pindborg tumor.

In one embodiment, the use of the compounds of Formula (I) for preparation of a medicament for the in vivo method of detection comprise compounds of formula (II):

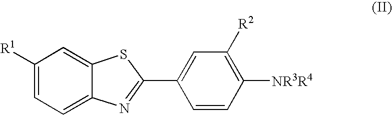

or a radiolabeled derivative, pharmaceutically acceptable salt, hydrate, solvate or prodrug of the compound, wherein:

R¹ is hydrogen, —OH, —NO₂, —CN, —COOR, —OCH₂OR, C₁-C₆ alkyl, C₂-C₆ alkenyl, C₂-C₆ alkynyl, C₁-C₆ alkoxy or halo;

R is C₁-C₆ alkyl;

R² is hydrogen or halo;

R³ is hydrogen, C₁-C₆ alkyl, C₂-C₆ alkenyl or C₂-C₆ alkynyl; and

R⁴ is hydrogen, C₁-C₆ alkyl, C₂-C₆ alkenyl or C₂-C₆ alkynyl, wherein the alkyl, alkenyl or alkynyl comprises a radioactive carbon or is substituted with a radioactive halo when R² is hydrogen or a non-radioactive halo;

provided that when R¹ is hydrogen or —OH, R² is hydrogen and R⁴ is —¹¹CH₃, then R³ is C₂-C₆ alkyl, C₂-C₆ alkenyl or C₂-C₆ alkynyl; and

further provided that when R¹ is hydrogen, R² hydrogen and R⁴ is —(CH₂)₃¹⁸F, then R³ is C₂-C₆ alkyl, C₂-C₆ alkenyl or C₂-C₆ alkynyl, where at least one of the substituent moieties comprises a detectable label.

In one embodiment, the use of the compounds of Formula (I) for preparation of a medicament for the in vivo method of detection comprise a compound selected from the group consisting of structures 1-45 or a radiolabeled derivative thereof, wherein the compound comprises at least one detectable label:

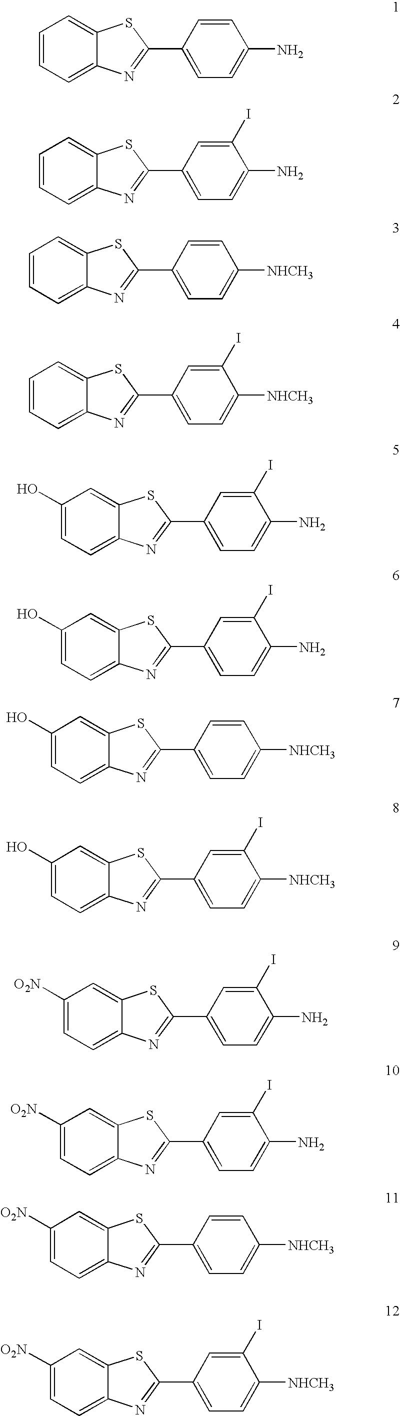

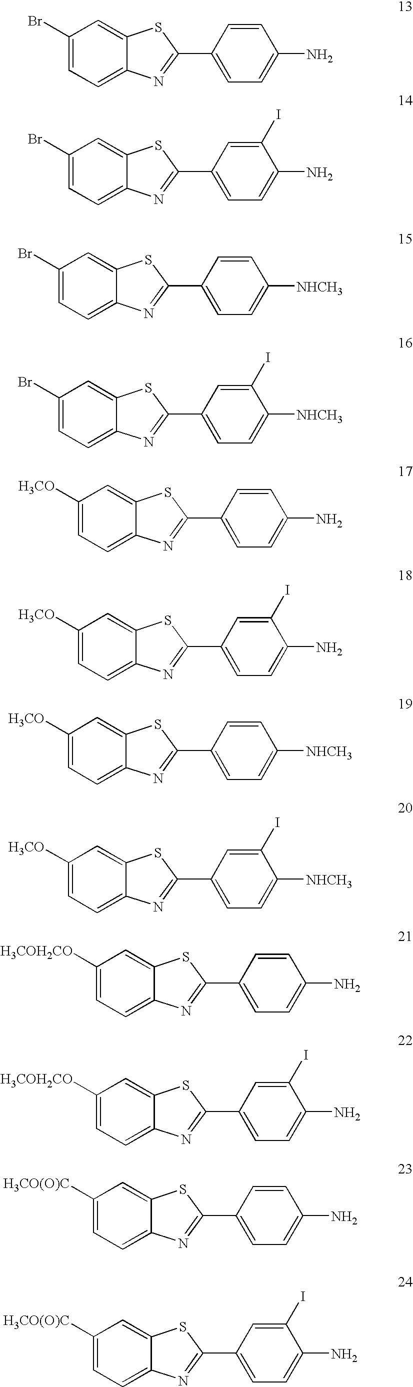

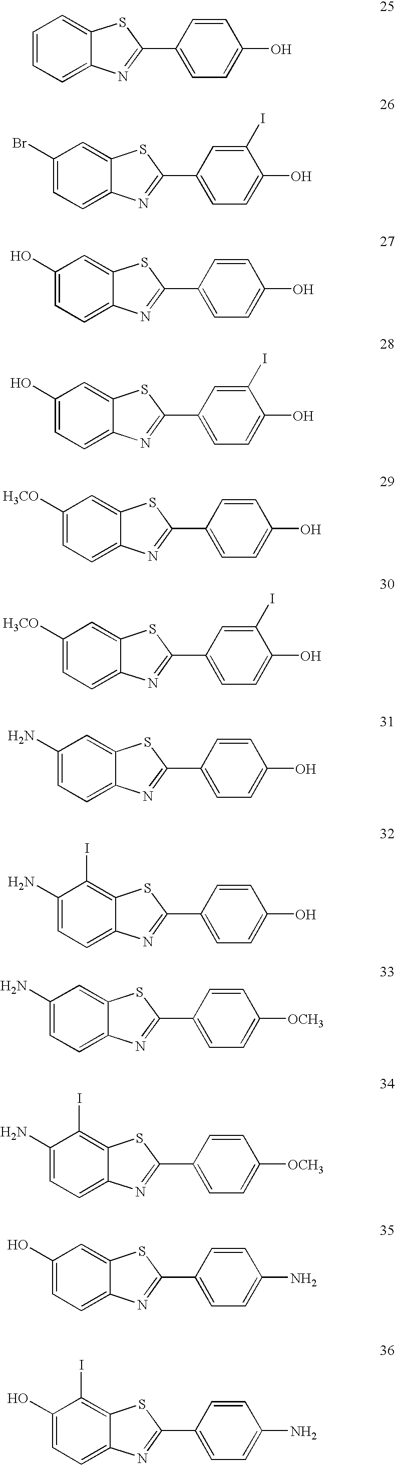

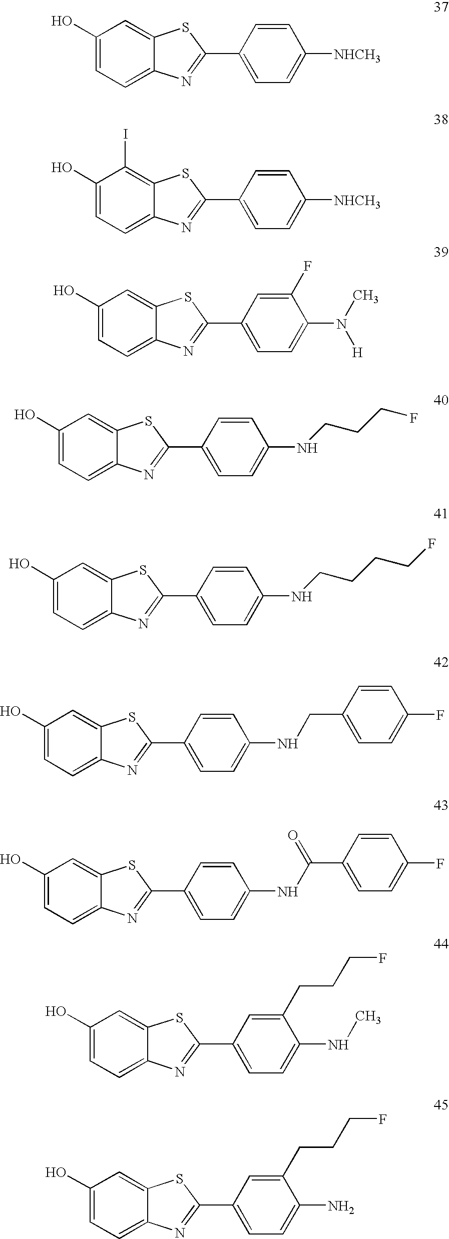

### Amyloid Probes

The amyloid probe of the present invention is any compound of formula (I), described above. In some embodiments, the amyloid probe is a compound of formula (II)

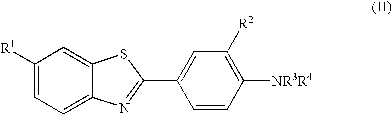

or a radiolabeled derivative, pharmaceutically acceptable salt, hydrate, solvate, or prodrug of the compound (II), wherein:

R¹ is hydrogen, —OH, —NO₂, —CN, —COOR, —OCH₂OR, C₁-C₆ alkyl, C₂-C₆ alkenyl, C₂-C₆ alkynyl, C₁-C₆ alkoxy or halo;

R is C₁-C₆ alkyl;

R² is hydrogen or halo;

R³ is hydrogen, C₁-C₆ alkyl, C₂-C₆ alkenyl or C₂-C₆ alkynyl; and

R⁴ is hydrogen, C₁-C₆ alkyl, C₂-C₆ alkenyl or C₂-C₆ alkynyl, wherein the alkyl, alkenyl or alkynyl comprises a radioactive carbon or is substituted with a radioactive halo when R² is hydrogen or a non-radioactive halo;
provided that when R¹ is hydrogen or —OH, R² is hydrogen and R⁴ is —¹CH₃, then R³ is C₂-C₆ alkyl, C₂-C₆ alkenyl or C₂-C₆ alkynyl; and
   further provided that when R¹ is hydrogen, R² hydrogen and R⁴ is —(CH₂)₃¹⁸F, then R³ is C₂-C₆ alkyl, C₂-C₆ alkenyl or C₂-C₆ alkynyl, where at least one of the substituent moieties comprises a detectable label.

In one embodiment, R² in the compounds of formula (II) contains a radioactive halo.

“Alkyl” refers to a saturated straight or branched chain hydrocarbon radical. Examples include without limitation methyl, ethyl, propyl, iso-propyl, butyl, iso-butyl, tert-butyl, n-pentyl and n-hexyl. The term “lower alkyl” refers to C₁-C₆ alkyl.

“Alkenyl” refers to an unsaturated straight or branched chain hydrocarbon radical comprising at least one carbon to carbon double bond. Examples include without limitation ethenyl, propenyl, iso-propenyl, butenyl, iso-butenyl, tert-butenyl, n-pentenyl and n-hexenyl.

“Alkynyl” refers to an unsaturated straight or branched chain hydrocarbon radical comprising at least one carbon to carbon triple bond. Examples include without limitation ethynyl, propynyl, iso-propynyl, butynyl, iso-butynyl, tert-butynyl, pentynyl and hexynyl.

“Alkoxy” refers to an alkyl group bonded through an oxygen linkage.

“Halo” refers to a fluoro, chloro, bromo or iodo radical.

“Radioactive halo” refers to a radioactive halo, i.e. radiofluoro, radiochloro, radiobromo or radioiodo.

In another embodiment, the thioflavin compound of formula (I) is selected from radiolabeled derivatives of one of structures 1-45:

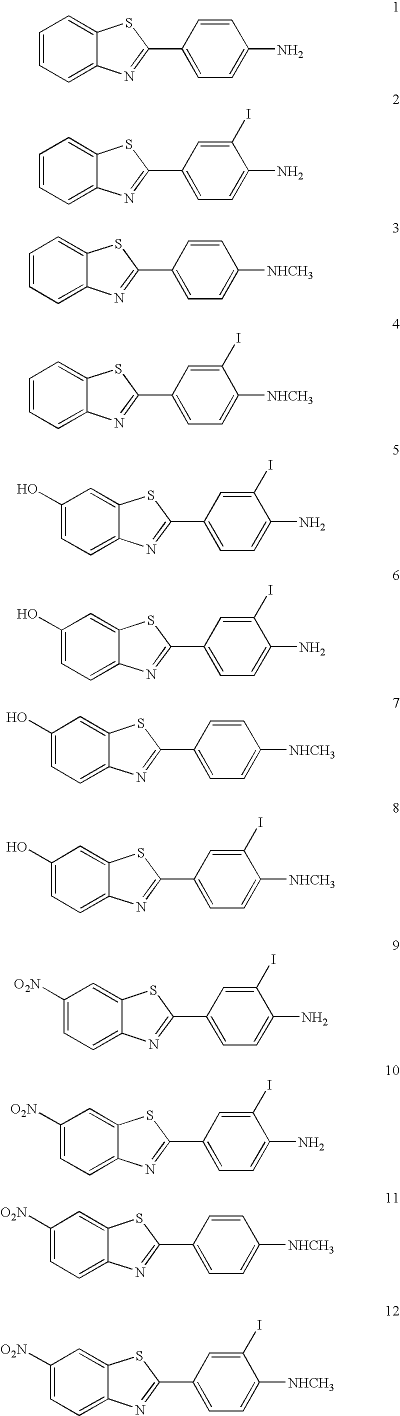

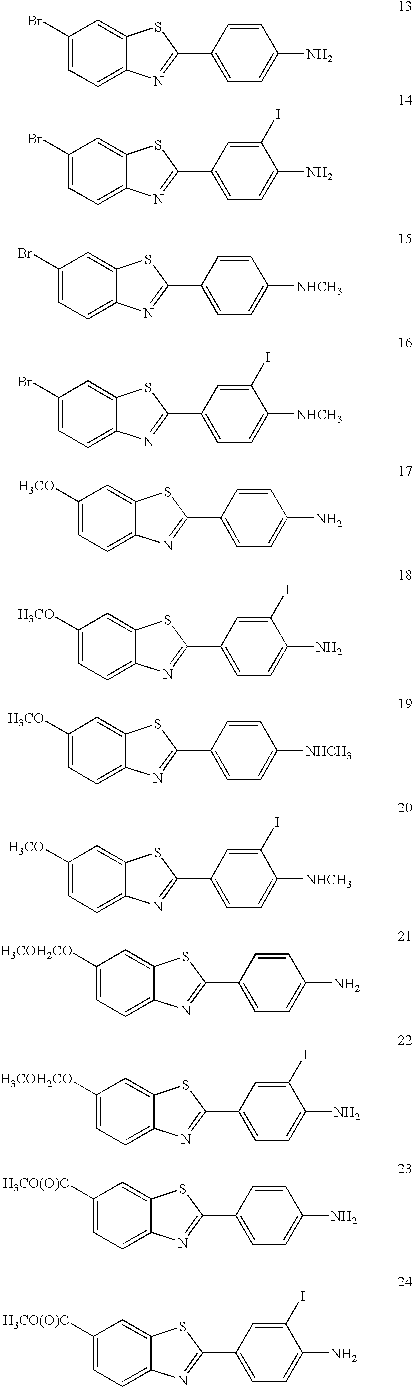

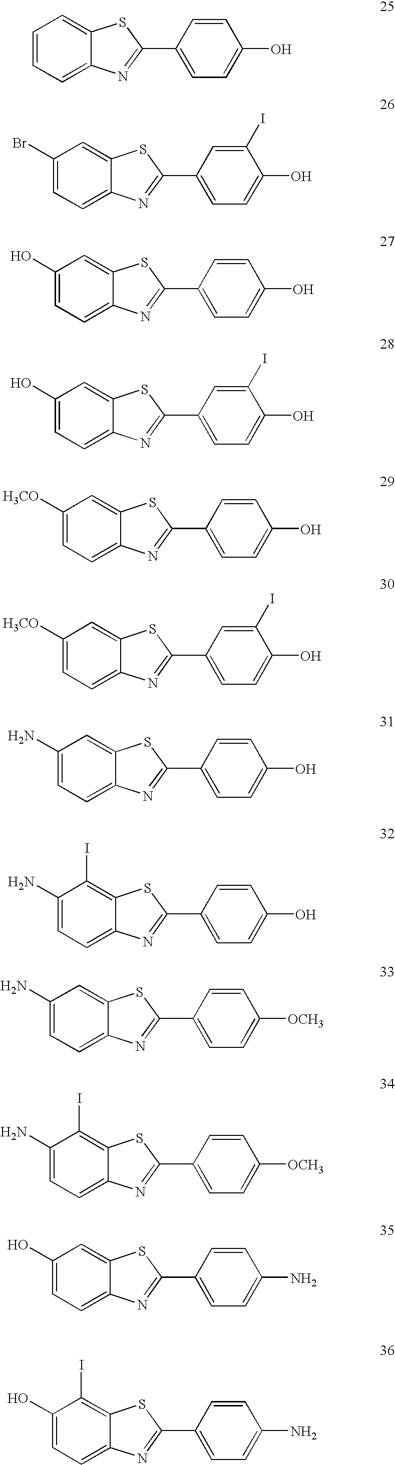

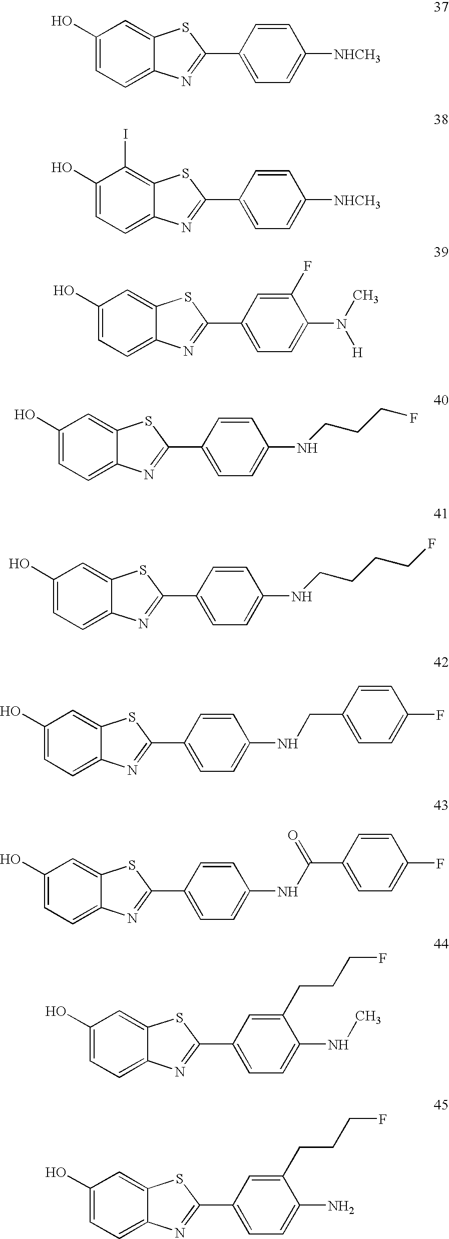

In preferred embodiments, the amyloid probe is {N-methyl-¹¹C}2-[4′-(methylamino)phenyl]6-hydroxybenzothiazole (“[¹¹C]PIB”) or {N-methyl-³H}2-[4′-(methylamino)phenyl]6-hydroxybenzothiazole (“[³]PIB”).

“Effective amount” refers to the amount required to produce a desired effect. Examples of an “effective amount” include amounts that enable detecting and imaging of amyloid deposit(s) in vivo or in vitro, that yield acceptable toxicity and bioavailability levels for pharmaceutical use, and/or prevent cell degeneration and toxicity associated with fibril formation.

Compounds of formulas (I), (II) and structures 1-45, also referred to herein as “thioflavin compounds,” “thioflavin derivatives,” or “amyloid probes,” have the following characteristic: specific binding an amyloid deposit which comprises at least one amyloidogenic protein, wherein the amyloidogenic protein is selected from the group consisting of AL, AH, ATTR, Aβ2M, AA, AApoAI, AApoAII, AGel, ALys, AFib, ACys, ABri, ADan, APrP, ACal, AlAPP, AANF, APro, AIns, AMed, AKer, A(tbn), and ALac.

The present compounds are non-quaternary amine derivatives of Thioflavin S and T which are known to stain amyloid in tissue sections and bind to synthetic Aβ in vitro. Kelenyi J. Histochem. Cytochem. 15: 172 (1967); Burns et al. J. Path. Bact. 94:337 (1967); Guntem et al. Experientia 48: 8 (1992); LeVine Meth. Enzylnol. 309: 274 (1999).

A method of this invention determines the presence and location of amyloid deposits in an organ or body area of a patient. The present method comprises administration of a detectable quantity of an amyloid probe of formulas (I) or (II) and structures 1-45. In some embodiments, the amyloid probe is chosen from structures 1-45, as shown above. An amyloid probe may be administered to a patient as a pharmaceutical composition or a pharmaceutically acceptable water-soluble salt thereof.

“Pharmaceutically acceptable salt” refers to an acid or base salt of the inventive compound, which salt possesses the desired pharmacological activity and is neither biologically nor otherwise undesirable. The salt can be formed with acids that include without limitation acetate, adipate, alginate, aspartate, benzoate, benzenesulfonate, bisulfate butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride hydrobromide, hydroiodide, 2-hydroxyethane-sulfonate, lactate, maleate, methanesulfonate, 2-naphthalenesulfonate, nicotinate, oxalate, thiocyanate, tosylate and undecanoate. Examples of a base salt include without limitation ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine and lysine. In some embodiments, the basic nitrogen-containing groups can be quarternized with agents including lower alkyl halides such as methyl, ethyl, propyl and butyl chlorides, bromides and iodides; dialkyl sulfates such as dimethyl, diethyl, dibutyl and diamyl sulfates; long chain halides such as decyl, lauryl, myristyl and stearyl chlorides, bromides and iodides; and aralkyl halides such as phenethyl bromides.

Generally, the dosage of the detectably labeled thioflavin derivative will vary depending on considerations such as age, condition, sex, and extent of disease in the patient, contraindications, if any, concomitant therapies and other variables, to be adjusted by a physician skilled in the art. Dosage can vary from 0.001 μg/kg to 10 μg/kg, preferably 0.01 μg/kg to 1.0 μg/kg.

Administration to the subject may be local or systemic and accomplished intravenously, intraarterially, intrathecally (via the spinal fluid) or the like. Administration may also be intradermal or intracavitary, depending upon the body site under examination. After a sufficient time has elapsed for the compound to bind with the amyloid, for example 30 minutes to 48 hours, the area of the subject under investigation is examined by routine imaging techniques such as MRS/MRI, SPECT, planar scintillation imaging, PET, as well as emerging imaging techniques. The exact protocol will necessarily vary depending upon factors specific to the patient, as noted above, and depending upon the body site under examination, method of administration and type of label used; the determination of specific procedures would be routine to the skilled artisan. For organ imaging, preferably, the amount (total or specific binding) of the bound radioactively labeled thioflavin derivative or analogue of the present invention is measured and compared (as a ratio) with the amount of labeled thioflavin derivative bound to the organ of the patient. This ratio is then compared to the same ratio in age-matched normal organ.

The radiolabelled amyloid probes will be injected intravenously. The PET scanning protocol would likely involve a standard whole body scan (covering from head to pelvis) completed 15-60 min after the injection of the radiopharmaceutical or a scan over a particular body area (e.g., heart, lungs, liver, kidneys). This scanning protocol likely would be analogous to a whole body or a focused body area PET oncology scan performed with [F-18]2-fluoro-2-deoxyglucose (FDG). That is, the amyloid-specific radiopharmaceutical is injected intravenously, time is alloted for radiotracer distribution throughout the body, radiotracer uptake in the organ(s) of interest, and clearance from the blood and other organs in which amyloid is absent, and a 20-40 min scan is performed over the whole body or over a particular body area to image amyloid-bound radiotracer. In addition, the imaging scan(s) can be used to subsequently direct biopsy sampling of the scanned tissue(s).

The amyloid probes of the present invention are advantageously administered in the form of injectable compositions, but may also be formulated into well known drug delivery systems (e.g., oral, rectal, parenteral (intravenous, intramuscular, or subcutaneous), intracisternal, intravaginal, intraperitoneal, local (powders, ointments or drops), or as a buccal or nasal spray). A typical composition for such purpose comprises a pharmaceutically acceptable carrier. For instance, the composition may contain about 10 mg of human serum albumin and from about 0.5 to 500 micrograms of the labeled thioflavin derivative per milliliter of phosphate buffer containing NaCl. Other pharmaceutically acceptable carriers include aqueous solutions, non-toxic excipients, including salts, preservatives, buffers and the like, as described, for instance, in REMINGTON'S PHARMACEUTICAL SCIENCES, 15th Ed. Easton: Mack Publishing Co. pp. 1405-1412 and 1461-1487 (1975) and THE NATIONAL FORMULARY XIV., 14th Ed. Washington: American Pharmaceutical Association (1975), the contents of which are hereby incorporated by reference.

Particularly preferred amyloid probes of the present invention are those that, in addition to specifically binding amyloid in vivo are also non-toxic at appropriate dosage levels and have a satisfactory duration of effect.

Examples of non-aqueous solvents are propylene glycol, polyethylene glycol, vegetable oil and injectable organic esters such as ethyl oleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial, anti-oxidants, chelating agents and inert gases. The pH and exact concentration of the various components the pharmaceutical composition are adjusted according to routine skills in the art. See, Goodman and Gilman's THE PHARMACOLOGICAL BASIS FOR THERAPEUTICS (7th Ed.).

According to the present invention, a pharmaceutical composition comprising an amyloid probe of formula (I) or formula (II) or one of the structures 1-45, is administered to subjects in whom amyloid or amyloid deposits are anticipated, e.g., patients clinically diagnosed a disease associated with amyloid deposition.

### Imaging

The invention employs amyloid probes which, in conjunction with non-invasive imaging techniques such as magnetic resonance spectroscopy (MRS) or imaging (MRI), or gamma imaging such as positron emission tomography (PET) or single-photon emission computed tomography (SPECT), are used to quantify amyloid deposition in vivo and in vitro.

The term “in vivo or in vitro method for detecting” refers to any method which permits the detection of a labeled thioflavin derivative of formulas (I) or (II) or one of structures 1-45. As an example, for gamma imaging, the radiation emitted from the organ or area being examined is measured and expressed either as total binding or as a ratio in which total binding in one tissue is normalized to (for example, divided by) the total binding in another tissue of the same subject during the same in vivo imaging procedure. Total binding in vivo is defined as the entire signal detected in a tissue by an in vivo imaging technique without the need for correction by a second injection of an identical quantity of labeled compound along with a large excess of unlabeled, but otherwise chemically identical compound. Similarly, in vitro methods would involve obtaining a fresh or frozen tissue specimen and incubating a section of the tissue or a homogenate of the tissue with a radioactively labeled thioflavin derivative of formulas (I) or (II) or one of structures 1-45, and then separating bound and free radiolabel by washing the tissue section or filtering and washing the tissue homogenate. The bound radioactivity is measured by standard autoradiographic techniques or by liquid scintillation or gamma counting and compared to controls from the same tissue to which an excess of unlabeled thioflavin derivative has been added.

A “subject” is a mammal, preferably a human, and most preferably a human suspected of having a disease associated with amyloid deposition, such as AD and/or dementia. The term “subject” and “patient” are used interchangeably herein.

For purposes of in vivo and in vitro imaging, the type of detection instrument available is a major factor in selecting a given label. For instance, radioactive isotopes and ¹⁸F are particularly suitable for in vivo and in vitro imaging in the methods of the present invention. The type of instrument used will guide the selection of the radionuclide or stable isotope. For instance, the radionuclide chosen must have a type of decay detectable by a given type of instrument. Another consideration relates to the half-life of the radionuclide. The half-life should be long enough so that it is still detectable at the time of maximum uptake by the target, but short enough so that the host does not sustain deleterious radiation. The radiolabeled compounds of the invention can be detected using gamma imaging wherein emitted gamma irradiation of the appropriate wavelength is detected. Methods of gamma imaging include, but are not limited to, SPECT and PET. Preferably, for SPECT detection, the chosen radiolabel will lack a particulate emission, but will produce a large number of photons in a 140-200 keV range. For PET detection, the radiolabel will be a positron-emitting radionuclide such as ¹⁸F which will annihilate to form two 511 keV gamma rays which will be detected by the PET camera.

In the present invention, amyloid binding compounds/probes, which are useful for in vivo and in vitro imaging and quantification of amyloid deposition, are administered to a patient. These compounds are to be used in conjunction with non-invasive neuroimaging techniques such as magnetic resonance spectroscopy (MRS) or imaging (MRI), positron emission tomography (PET), and single-photon emission computed tomography (SPECT). In accordance with this invention, the thioflavin derivatives may be labeled with ¹⁸F or ¹³C for MRS/MRI by general organic chemistry techniques known to the art. See, e.g., March, J. ADVANCED ORGANIC CHEMISTRY: REACTIONS, MECHANISMS, AND STRUCTURE (3rd Edition, 1985), the contents of which are hereby incorporated by reference. The thioflavin derivatives also may be radiolabeled with ¹⁸F, ¹¹C, ⁷⁵Br, or ⁷⁶Br for PET by techniques well known in the art and are described by Fowler, J. and Wolf, A. in POSITRON EMISSION TOMOGRAPHY AND AUTORADIOGRAPHY (Phelps, M., Mazziota, J., and Schelbert, H. eds.) 391-450 (Raven Press, NY 1986) the contents of which are hereby incorporated by reference. The thioflavin derivatives also may be radiolabeled with ¹²³I for SPECT by any of several techniques known to the art. See, e.g., Kulkami, Int. J. Rad. Appl. & Inst. (Part B) 18: 647 (1991), the contents of which are hereby incorporated by reference. In addition, the thioflavin derivatives may be labeled with any suitable radioactive iodine isotope, such as, but not limited to ¹³¹I, ¹²⁵I, or ¹²³I, by iodination of a diazotized amino derivative directly via a diazonium iodide, see Greenbaum, F. Am. J. Pharm. 108: 17 (1936), or by conversion of the unstable diazotized amine to the stable triazene, or by conversion of a non-radioactive halogenated precursor to a stable tri-alkyl tin derivative which then can be converted to the iodo compound by several methods well known to the art. See, Satyamurthy and Barrio J. Org. Chem. 48: 4394 (1983), Goodman et al., J. Org. Chem. 49: 2322 (1984), and Mathis et al., J. Labell. Comp. and Radiopharm. 1994: 905; Chumpradit et al., J. Med. Chem. 34: 877 (1991); Zhuang et al., J. Med. Chem. 37: 1406 (1994); Chumpradit et al., J. Med. Chem. 37: 4245 (1994). For example, a stable triazene or tri-alkyl tin derivative of thioflavin or its analogues is reacted with a halogenating agent containing ¹³¹I, ¹²⁵I, ¹²³I, ⁷⁶Br, ⁷⁵Br, ¹⁸F or ¹⁹F. Thus, the stable tri-alkyl tin derivatives of thioflavin and its analogues are novel precursors useful for the synthesis of many of the radiolabeled compounds within the present invention. As such, these tri-alkyl tin derivatives are one embodiment of this invention.

The thioflavin derivatives also may be radiolabeled with known metal radiolabels, such as Technetium-99m (^{99m}Tc). Modification of the substituents to introduce ligands that bind such metal ions can be effected without undue experimentation by one of ordinary skill in the radiolabeling art. The metal radiolabeled thioflavin derivative can then be used to detect amyloid deposits. Preparing radiolabeled derivatives of Tc^{99m} is well known in the art. See, for example, Zhuang et al., “Neutral and stereospecific Tc-99m complexes: [99mTc]N-benzyl-3,4-di-(N-2-mercaptoethyl)-amino-pyrrolidines (P-BAT)” Nuclear Medicine & Biology 26(2):217-24, (1999); Oya et al., “Small and neutral Tc(v)O BAT, bisaminoethanethiol (N2S2) complexes for developing new brain imaging agents” Nuclear Medicine & Biology 25(2): 135-40, (1998); and Hom et al., “Technetium-99m-labeled receptor-specific small-molecule radiopharmaceuticals: recent developments and encouraging results” Nuclear Medicine & Biology 24(6):485-98, (1997).

The methods of the present invention may use isotopes detectable by nuclear magnetic resonance spectroscopy for purposes of in vivo or in vitro imaging and spectroscopy. Elements particularly useful in magnetic resonance spectroscopy include ¹⁸F and ¹³C.

Suitable radioisotopes for purposes of this invention include beta-emitters, gamma-emitters, positron-emitters, and x-ray emitters. These radioisotopes include ¹³¹I, ¹²³I, ¹⁸F, ¹¹C, ⁷⁵Br, and ⁷⁶Br. Suitable stable isotopes for use in Magnetic Resonance Imaging (MRI) or Spectroscopy (MRS), according to this invention, include ¹⁸F and ¹³C. Suitable radioisotopes for in vitro quantification of amyloid in homogenates of biopsy or post-mortem tissue include ¹²⁵I, ¹⁴C, and ³H. The preferred radiolabels are ¹¹C or ¹⁸F for use in PET in vivo imaging, ¹²³I for use in SPECT imaging, ¹⁹F for MRS/MRI, and ³H or ¹⁴C for in vitro studies. However, any conventional method for visualizing diagnostic probes as have accumulated in targets to a detectable level can be utilized in accordance with this invention.

According to an aspect of the invention which relates to a method of detecting amyloid deposits in biopsy tissue, the method involves incubating formalin-fixed tissue with a solution of a thioflavin amyloid binding compound chosen from compounds of formulas (I) and (II) or structures 1-45, described above. Preferably, the solution is 25-100% ethanol, (with the remainder being water) saturated with a thioflavin amyloid binding compound of formulas (I) or (II) or structures 1-45 according to the invention. Upon incubation, the compound stains or labels the amyloid deposit in the tissue, and the stained or labeled deposit can be detected or visualized by any standard method. Such detection means include microscopic techniques such as bright-field, fluorescence, laser-confocal and cross-polarization microscopy.

The method of quantifying the amount of amyloid in biopsy tissue involves incubating, with homogenate of biopsy or post-mortem tissue, a labeled derivative of thioflavin, according to the present invention, or a water-soluble, non-toxic salt thereof. The tissue is obtained and homogenized by well-known techniques. The preferred label is a radiolabel, although other, suitable labels are available, such as enzymes, chemiluminescent, and immunofluorescent compounds. The preferred radiolabel is ¹²⁵I, ¹⁴C or ³H, which is contained in a substituent substituted on one of the compounds of formulas (I) or (II) or structures 1-45. Tissue containing amyloid deposits will bind to the labeled derivatives of the amyloid-binding thioflavin compounds of the present invention. The bound tissue then is separated from the unbound tissue by any conventional means, such as filtering. The bound tissue then can be quantified via any of a variety of known approaches. The units of tissue-bound, radiolabeled thioflavin derivative then are converted to units of micrograms of amyloid per 100 mg of tissue, by comparison to a standard curve generated by incubating known amounts of amyloid with the radiolabeled thioflavin derivative.

As described above, the specific method of detection will vary, depending upon the chemical and physical nature of the species utilized and detected. Thus, for gamma-emitting species, standard, commercially available single photon and positron detection methods can be utilized. For magnetic nuclear spin detection, standard, commercially available magnetic resonance imaging and spectroscopy techniques can be utilized.

In the methods herein described, data collection using these technologies are conducted according to standard clinical imaging protocols involving whole body imaging techniques, such as repeatedly moving the subject through the scanner over the course of the scanning period. Alternatively, data collection may be achieved by imaging selectively over one or more regions of interest in the body, for example by emphasizing the lungs, liver, heart or kidneys using a limited range of patient body coverage in an imaging scanner. Following the administration of a compound of formula (I), imaging data collection can begin immediately and proceed for several hours post administration using a dynamic imaging protocol. Alternatively, late-time snapshots of about 30 minutes could be taken following the in vivo distribution of the compound using standard static late time imaging protocols. Imaging data then is collected and stored electronically in an automated and routine fashion, for later processing and analysis.

Data processing and analysis typically make use of commercially available software packages, which often are installed by the manufacturer on the single photon, positron emission, or magnetic resonance scanners' operating system computers. Examples of these processes and methods for detecting, collecting, and processing imaging data are established in the art for positron emission methodologies (see J. C. Price et al., “Kinetic modeling of amyloid binding in humans using PET imaging and Pittsburgh Compound-B,” 25 J. Cerebral Blood Flow and Metabolism (2005) 1528-47 and B. J. Lopresti et al. “Simplified Quantification of Pittsburgh Compound-B Amyloid Imaging PET Studies: A Comparative Analysis,” 46 J. Nuclear Medicine (2005) 1959-72. Analogous data collection and processing of single photon, positron, and magnetic resonance species are similarly conducted for systemic amyloid deposits using standard, commercially available scanners, data collection methodologies, and data processing techniques in body regions outside the brain.

Unless the context clearly dictates otherwise, the definitions of singular terms may be extrapolated to apply to their plural counterparts as they appear in the application; likewise, the definitions of plural terms may be extrapolated to apply to their singular counterparts as they appear in the application.

The following examples are given to illustrate the present invention. It should be understood, however, that the invention is not to be limited to the specific conditions or details described in these examples. Throughout the specification, any and all references to a publicly available document, including U.S. patents, are specifically incorporated into this patent application by reference.

### SYNTHETIC EXAMPLES

Compounds of formulas (I) and (II), and the formulae of structures 1-45, can be prepared by methods that are well known in the art. See, e.g., WO 2002/16333, U.S. Patent Publication No. 2003/0236391, published Dec. 25, 2003, and WO 2004/083195, the entire contents of which are herein incorporated by reference.

All of the reagents used in the synthesis were purchased from Aldrich Chemical Company and used without further purification, unless otherwise indicated. Melting points were determined on MeI-TEMP II and were uncorrected. The ¹H NMR spectra of all compounds were measured on Bruker 300 using TMS as internal reference and were in agreement with the assigned structures. The TLC was performed using Silica Gel 60 F₂₅₄ from EM Sciences and detected under UV lamp. Flash chromatography was performed on silica gel 60 (230-400 mesh. Purchased from Mallinckrodt Company. The reverse phase TLC were purchased from Whiteman Company.

### General Methodology for Synthesis of Compound of Formula (I):

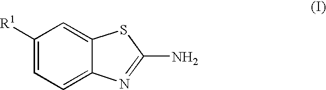

R¹ is hydrogen, —OH, —NO₂, —CN, —COOR, —OCH₂OR, C₁-C₆ alkyl, C₂-C₆ alkenyl, C₂-C₆ alkynyl, C₁-C₆ alkoxy or halo, wherein one or more of the atoms of R¹ may be a radiolabeled atom;

R is C₁-C₆ alkyl, wherein one or more of the carbon atoms may be a radiolabeled atom;

is hydrolysed by one of the following two procedures:

### Preparation of 2-aminothiophenol Via Hydrolysis

The 6-substituted 2-aminobenzothiazole (172 mmol) is suspended in 50% KOH (180 g KOH dissolved in 180 mL water) and ethylene glycol (40 mL). The suspension is heated to reflux for 48 hours. Upon cooling to room temperature, toluene (300 mL) is added and the reaction mixture is neutralized with acetic acid (180 mL). The organic layer is separated and the aqueous layer is extracted with another 200 mL of toluene. The toluene layers are combined and washed with water and dried over MgSO₄. Evaporation of the solvent gives the desired product.

### Preparation of 2-aminothiophenol via Hydrazinolysis

The 6-substituted-benzothiazole (6.7 mmol) is suspended in ethanol (11 mL, anhydrous) and hydrazine (2.4 mL) is added under a nitrogen atmosphere at room temperature. The reaction mixture is heated to reflux for 1 hour. The solvent is evaporated and the residue is dissolved into water (10 mL) and adjusted to a pH of 5 with acetic acid. The precipitate is collected with filtration and washed with water to give the desired product.

The resulting 5-substituted-2-amino-1-thiophenol of the form

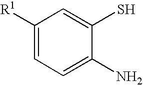

is coupled to a benzoic acid of the form:

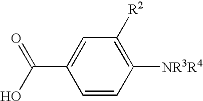

wherein R² is hydrogen, and R³ and R⁴ are independently hydrogen, C₁-C₆ alkyl, C₂-C₆ alkenyl or C₂-C₆ alkynyl

by the following methodology:

A mixture of the 5-substituted 2-aminothiophenol (4.0 mmol), the benzoic acid (4.0 mmol), and polyphosphoric acid (PPA) (10 g) is heated to 220° C. for 4 hours. The reaction mixture is cooled to room temperature and poured into 10% potassium carbonate solution (˜400 mL). The precipitate is collected by filtration under reduced pressure to give the desired product, which can be purified by flash chromatography or recrystallization.

The R² hydrogen can be substituted with either a non-radioactive halo or a radioactive halo by the following reaction:

To a solution of 6-substituted 2-(4′-aminophenyl)-benzothiazole (1 mg) in 250 μL acetic acid in a sealed vial is added 40 μL of chloramine-T solution (28 mg dissolved in 500 μL acetic acid) followed by 27 μL (ca. 5 mCi) of sodium [¹²⁵I]iodide (specific activity 2,175 Ci/mmol). The reaction mixture is stirred at room temperature for 2.5 hours and quenched with saturated sodium hydrogensulfite solution. After dilution with 20 ml of water, the reaction mixture is loaded onto C8 Plus SepPak and eluted with 2 ml methanol. Depending on the nature of the substituent on the 6-position, protecting groups may need to be employed. For example, the 6-hydroxy group is protected as the methanesulfonyl (mesyloxy) derivative. For deprotection of the methanesulfonyl group, 0.5 ml of 1 M NaOH is added to the eluted solution of radioiodinated intermediate. The mixture is heated at 50° C. for 2 hours. After being quenched by 500 μL of 1 M acetic acid, the reaction mixture is diluted with 40 mL of water and loaded onto a C8 Plus SepPak. The radioiodinated product, having a radioactivity of ca. 3 mCi, is eluted off the SepPak with 2 mL of methanol. The solution is condensed by a nitrogen stream to 300 μL and the crude product is purified by HPLC on a Phenomenex ODS column (MeCN/TEA buffer, 35:65, pH 7.5, flow rate 0.5 mL/minute up to 4 minutes, 1.0 mL/minute at 4-6 minutes, and 2.0 mL/minute after 6 minutes, retention time 23.6). The collected fractions are loaded onto a C8 Plus SepPak. Elution with 1 mL of ethanol gave ca. 1 mCi of the final radioiodinated product.

When either or both R³ and R⁴ are hydrogen, then R³ and R⁴ can be converted to C₁-C₆ alkyl, C₂-C₆ alkenyl or C₂-C₆ alkynyl by reaction with an alkyl, alkenyl or alkynyl halide under the following conditions:

For dialkylation: To a solution of 6-substituted 2-(4′-aminophenyl)-benzothiazole (0.59 mmol) in DMSO (anhydrous, 2 ml) are added alkyl, alkenyl, or alkynyl halide (2.09 mmol), and K₂CO₃ (500 mg, 3.75 mmol). The reaction mixture is heated at 140° C. for 16 hours. Upon cooling to room temperature, the reaction mixture is poured into water and extracted with ethyl acetate (3×10 mL). The organic layers are combined and the solvent is evaporated. The residue is purified by flash column to give the desired 6-substituted dimethylaminophenyl)-benzothiazole.

For monoalkylation: To a solution of 6-substituted 2-(4′-aminophenyl)-benzothiazole (0.013 mmol) in DMSO (anhydrous, 0.5 ml) is added alkyl, alkenyl, or alkynyl halide (0.027 mmol) and anhydrous K₂CO₃ (100 mg, 0.75 mmol). The reaction mixture is heated at 100° C. for 16 hours. Upon cooling to room temperature, the reaction mixture is directly purified by normal phase preparative TLC to give the desired 6-substituted-2-(4′-methylaminophenyl)-benzothiazole derivatives.

When R² is hydrogen or a non-radioactive halo, R⁴ is C₁-C₆ alkyl, C₂-C₆ alkenyl or C₂-C₆ alkynyl, wherein the alkyl, alkenyl or alkynyl comprises a radioactive carbon or is substituted with a radioactive halo, the compound can be synthesized by one of the following sequences:

### For Radioactive Carbon Incorporation:

Approximately 1 Ci of [¹¹C]carbon dioxide is produced using a CTI/Siemens RDS 112 negative ion cyclotron by irradiation of a nitrogen gas (¹⁴N₂) target containing 1% oxygen gas with a 40 μA beam current of 11 MeV protons for 60 minutes. [¹¹C]Carbon dioxide is converted to [¹¹C]methyl iodide by first reacting it with a saturated solution of lithium aluminum hydride in THF followed by the addition of hydriodic acid at reflux temperature to generate [¹¹C]methyl iodide. The [¹¹C]methyl iodide is carried in a stream of nitrogen gas to a reaction vial containing the precursor for radiolabeling. The precursor, 6-substituted 2-(4′-aminophenyl)-benzothiazole (˜3.7 μmoles), is dissolved in 400 μL of DMSO. Dry KOH (10 mg) is added, and the 3 mL V-vial is vortexed for 5 minutes. No-carrier-added [¹¹C]methyl iodide is bubbled through the solution at 30 mL/minute at room temperature. The reaction is heated for 5 minutes at 95° C. using an oil bath. The reaction product is purified by semi-preparative HPLC using a Prodigy ODS-Prep column eluted with 60% acetonitrile/40% triethylammonium phosphate buffer pH 7.2 (flow at 5 mL/minute for 0-7 minutes then increased to 15 mL/minute for 7-30 minutes). The fraction containing [N-methyl-¹¹C] 6-substituted 2-(4′-methylaminophenyl)-benzothiazole (at about 15 min) is collected and diluted with 50 mL of water and eluted through a Waters C18 SepPak Plus cartridge. The C18 SepPak is washed with 10 mL of water, and the product is eluted with 1 mL of ethanol (absolute) into a sterile vial followed by 14 mL of saline. Radiochemical and chemical purities are >95% as determined by analytical HPLC (k′=4.4 using the Prodigy ODS (3) analytical column eluted with 65/35 acetonitrile/triethylammonium phosphate buffer pH 7.2). The radiochemical yield averages 17% at EOS based on [¹¹C]methyl iodide, and the specific activity averages about 160 GBq/μmol (4.3 Ci/μmol) at end of synthesis.

### For Radioactive Halogen Incorporation:

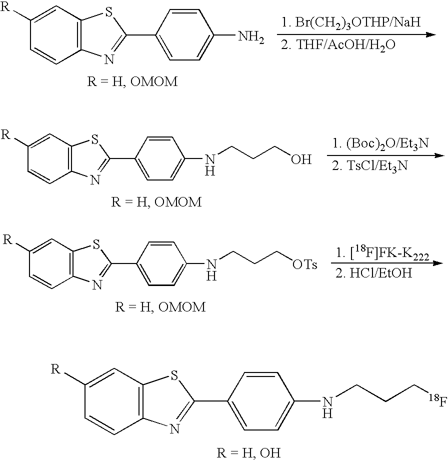

A mixture of 6-substituted 2-(4′-aminophenyl)-benzathiazole (protecting groups may be necessary depending on the nature of the 6-substituent as noted above) (0.22 mmol), NaH (4.2 mmol) and 2-(−3-bromopropoxy)tetrahydro-2-H-pyran (0.22 mmol) in THF (8 mL) is heated to reflux for 23 hours. The solvent is removed by distillation and the residue is dissolved in to ethyl acetate and water, the organic layer is separated and the aqueous layer is extracted with ethyl acetate (10 mL×6). The organic layer is combined and dried over MgSO₄ and evaporated to dryness. The residue is added AcOH/THF/H₂O solution (5 mL, 4/2/1) and heated to 100° C. for 4 hours. The solvent is removed by evaporation and the residue is dissolved in ethyl acetate (˜10 mL) washed by NaHCO₃ solution, dried over MgSO₄ and evaporated to dryness to give a residue which is purified with preparative TLC(hexane:ethyl acetate=60:40) to give the desired 6-substituted 2-(4′-(3″-hydroxypropylamino)-phenyl)-benzothiazole (45%).

To a solution of 6-substituted 2-(4′-(3″-hydroxypropylamino)-phenyl)-benzathiazole (0.052 mmol) and Et₃N (0.5 ml) dissolved in acetone (5 mL) is added (Boc)₂O (50 mg, 0.22 mmol). The reaction mixture is stirred at room temperature for 6 hours followed by addition of tosyl chloride (20 mg, 0.11 mmol). The reaction mixture is stirred at room temperature for another 24 hours. The solvent is removed and the residue is dissolved into ethyl acetate (10 mL), washed with NaCO₃ solution, dried over MgSO₄, evaporated, and purified with flash column (Hexane/ethyl acetate=4/1) to give the desired 6-substituted 2-(4′-(3″-toluenesulfonoxypropylamino)-phenyl)-benzothiazole (13%). This 6-substituted 2-(4′-(3″-toluenesulfonoxypropylamino)-phenyl)-benzothiazole is then radiofluorinated by standard methods as follows:

A cyclotron target containing 0.35 mL of 95% [O-18]-enriched water is irradiated with 11 MeV protons at 20 μA of beam current for 60 minutes, and the contents are transferred to a 5 mL reaction vial containing Kryptofix 222 (22.3 mg) and K₂CO₃ (7.9 mg) in acetonitrile (57 μL). The solution is evaporated to dryness three times at 110° C. under a stream of argon following the addition of 1 mL aliquots of acetonitrile. To the dried [F-18]fluoride is added 3 mg of 6-substituted 2-(4′-(3″-toluenesulfonoxypropylamino)-phenyl)-benzothiazole in 1 mL DMSO, and the reaction vial is sealed and heated to 85° C. for 30 minutes. To the reaction vial, 0.5 mL of MeOH/HCl (concentrated) (2/1 v/v) is added, and the vial is heated at 120° C. for 10 minutes. After heating, 0.3 mL of 2 M sodium acetate buffer is added to the reaction solution followed by purification by semi-prep HPLC using a Phenomenex Prodigy ODS-prep C18 column (10 μm 250×10 mm) eluted with 40% acetonitrile/60% 60 mM triethylamine-phosphate buffer (v/v) pH 7.2 at a flow rate of 5 mL/minute for 15 minutes, then the flow is increased to 8 mL/minute for the remainder of the separation. The product, [F-18]6-substituted 2-(4′-(3″-fluoropropylamino)-phenyl)-benzothiazole, is eluted at ˜20 minutes in a volume of about 16 mL. The fraction containing [F-18]6-substituted 2-(4′-(3″-fluoropropylamino)-phenyl)-benzothiazole is diluted with 50 mL of water and eluted through a Waters C18 SepPak Plus cartridge. The SepPak cartridge is then washed with 10 mL of water, and the product is eluted using 1 mL of ethanol (absol.) into a sterile vial. The solution is diluted with 10 mL of sterile normal saline for intravenous injection into animals. The [F-18]6-substituted 2-(4′-(3″-fluoropropylamino)-phenyl)-benzothiazole product is obtained in 2-12% radiochemical yield at the end of the 120 minute radiosynthesis (not decay corrected) with an average specific activity of 1500 Ci/mmol.

### Example 1

### [N-Methyl-¹¹C]2-(4′-Dimethylaminophenyl)-6-methoxy-benzothiazole was synthesized according to Scheme I

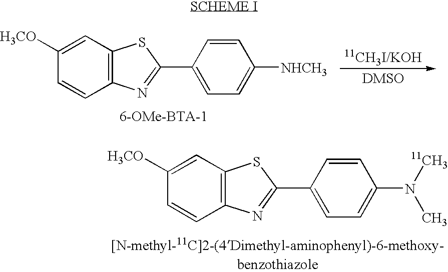

Approximately 1 Ci of [¹¹C]carbon dioxide was produced using a CTI/Siemens RDS 112 negative ion cyclotron by irradiation of a nitrogen gas (¹⁴N₂) target containing 1% oxygen gas with a 40 μA beam current of 11 MeV protons for 60 minutes. [¹¹C]Carbon dioxide is converted to [¹¹C]methyl iodide by first reacting it with a saturated solution of lithium aluminum hydride in THF followed by the addition of hydriodic acid at reflux temperature to generate [¹¹C]methyl iodide. The [¹¹C]methyl iodide is carried in stream of nitrogen gas to a reaction vial containing the precursor for radiolabeling. The precursor, 6-CH₃O-BTA-1 (1.0 mg, 3.7 μmoles), was dissolved in 400 μL of DMSO. Dry KOH (10 mg) was added, and the 3 mL V-vial was vortexed for 5 minutes. No-carrier-added [¹¹C]methyl iodide was bubbled through the solution at 30 mL/minute at room temperature. The reaction was heated for 5 minutes at 95° C. using an oil bath. The reaction product was purified by semi-preparative HPLC using a Prodigy ODS-Prep column eluted with 60% acetonitrile/40% triethylammonium phosphate buffer pH 7.2 (flow at 5 mL/minute for 0-7 minutes then increased to 15 mL/minute for 7-30 minutes). The fraction containing [N-Methyl-¹¹C]2-(4′-Dimethylaminophenyl)-6-methoxy-benzothiazole (at about 15 minutes) was collected and diluted with 50 mL of water and eluted through a Waters C18 SepPak Plus cartridge. The C18 SepPak was washed with 10 mL of water, and the product was eluted with 1 mL of ethanol (absolute) into a sterile vial followed by 14 mL of saline. Radiochemical and chemical purities were >95% as determined by analytical HPLC (k′=4.4 using the Prodigy ODS (3) analytical column eluted with 65/35 acetonitrile/triethylammonium phosphate buffer pH 7.2). The radiochemical yield averaged 17% at EOS based on [¹¹C]methyl iodide, and the specific activity averaged about 160 GBq/μmol (4.3 Ci/μmol) at end of synthesis.

### Example 2

### 2-(3′-¹²⁵I-iodo-4′-amino-phenyl)-benzothiazol-6-ol was synthesized according to Scheme II

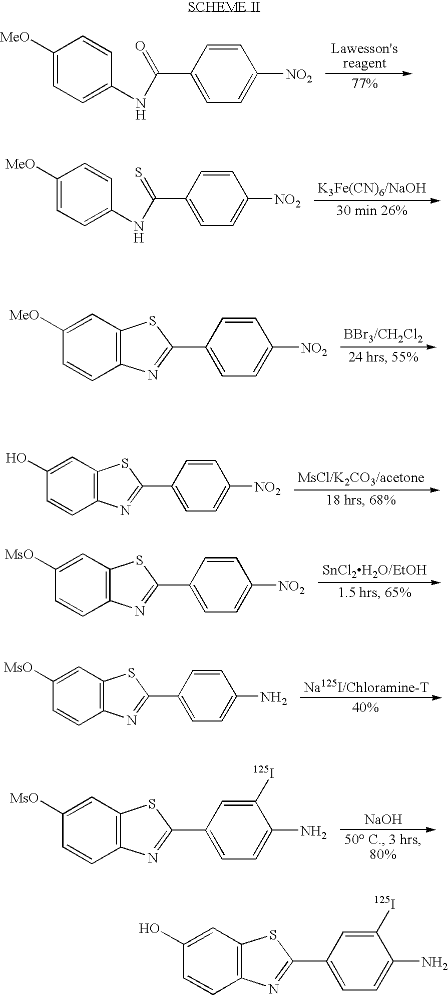

To a solution of 2-(4′-aminophenyl)-6-methanesulfonoxy-benzothiazole (1 mg) in 250 μL acetic acid in a sealed vial was added 40 μL of chloramine T solution (28 mg dissolved in 500 μL acetic acid) followed by 27 μL (ca. 5 mCi) of sodium [¹²⁵I]iodide (specific activity 2,175 Ci/mmol). The reaction mixture was stirred at room temperature for 2.5 hours and quenched with saturated sodium hydrogensulfite solution. After dilution with 20 ml of water, the reaction mixture was loaded onto C8 Plus SepPak and eluted with 2 ml methanol. For deprotection of the methaiesulfonyl group, 0.5 ml of 1 M NaOH was added to the eluted solution of radioiodinated intermediate. The mixture was heated at 50° C. for 2 hours. After being quenched by 500 μL of 1 M acetic acid, the reaction mixture was diluted with 40 mL of water and loaded onto a C8 Plus SepPak. The radioiodinated product, having a radioactivity of ca. 3 mCi, was eluted off the SepPak with 2 mL of methanol. The solution was condensed by a nitrogen stream to 300 μL and the crude product was purified by HPLC on a Phenomenex ODS column (MeCN/TEA buffer, 35:65, pH 7.5, flow rate 0.5 mL/minute up to 4 minutes, 1.0 mL/minute at 4-6 minutes, and 2.0 mL/minute after 6 minutes, retention time 23.6). The collected fractions were loaded onto a C8 Plus SepPak. Elution with 1 mL of ethanol gave ca. 1 mCi of the final radioiodinated product.

Preparation of the ¹²³I radiolabeled derivatives, proceeds similarly to the synthesis outlined above. For example, replacing sodium [¹²⁵I]iodide with sodium [¹²³I]iodide in the synthetic method would provide the ¹²³I radiolabeled compound. Such substitution of one radiohalo atom for another is well known in the art, see for example, Mathis C A, Taylor S E, Biegon A, Enas J D. [¹²⁵I]5-Iodo-6-nitroquipazine: a potent and selective ligand for the 5-hydroxytryptamine uptake complex I. In vitro studies. Brain Research 1993; 619:229-235; Jagust W, Eberling J L, Roberts J A, Bremian K M, Hanrahan S M, Van Brocklin H, Biegon A, Mathis C A. In vivo imaging of the 5-hydroxytryptamine reuptake site in primate brain using SPECT and [¹²³I]5-iodo-6-nitroquipazine. European Journal of Pharmacology 1993; 242:189-193; Jagust W J, Eberling J L, Biegon A, Taylor S E, VanBrocklin H, Jordan S, Hanrahan S M, Roberts J A, Brennan K M, Mathis C A. [Iodine-123]5-Iodo-6-Nitroquipazine: SPECT Radiotracer to Image the Serotonin Transporter. Journal of Nuclear Medicine 1996; 37:1207-1214.)

### Example 3

### 2-(3-¹⁸F-Fluoro-4-methylamino-phenyl)-benzothiazol-6-ol was synthesized according to Scheme III

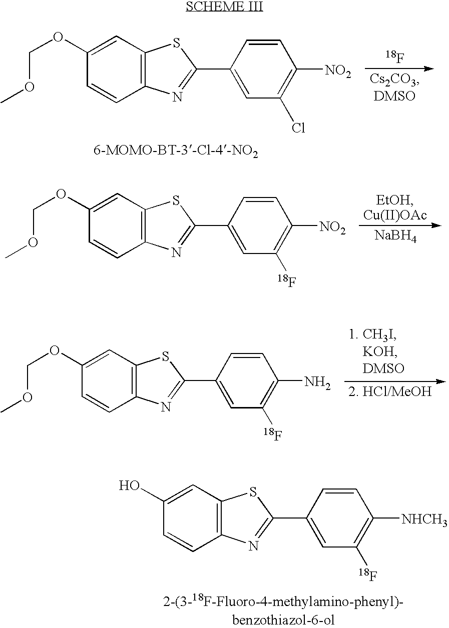

A cyclotron target containing 0.35 mL of 95% [O-18]-enriched water was irradiated with 11 MeV protons at 20 μA of beam current for 60 minutes, and the contents were transferred to a 5 mL reaction vial containing 2 mg Cs₂CO₃ in acetonitrile (57 μL). The solution was evaporated to dryness at 110° C. under a stream of argon three times using 1 mL aliquots of acetonitrile. To the dried [F-18]fluoride was added 6 mg of 6-MOMO-BT-3′-Cl-4′-NO₂ in 1 mL DMSO, and the reaction vial was sealed and heated to 120° C. for 20 minutes (radiochemical incorporation for this first radiosynthesis step was about 20% of solubilized [F-18]fluoride). To the crude reaction mixture was added 8 mL of water and 6 mL of diethyl ether, the mixture was shaken and allowed to separate. The ether phase was removed and evaporated to dryness under a stream of argon at 120° C. To the dried sample, 0.5 mL of absolute EtOH was added along with 3 mg copper (II) acetate and 8 mg of NaBH₄. The reduction reaction was allowed to proceed for 10 minutes at room temperature (the crude yield for the reduction step was about 40%). To the reaction mixture was added 8 mL of water and 6 mL of diethyl ether, the mixture was shaken and the ether phase separated. The diethyl ether phase was dried under a stream of argon at 120° C. To the reaction vial, 700 uL of DMSO was added containing 30 micromoles of CH₃I and 20 mg of dry KOH. The reaction vial was heated at 120° C. for 10 minutes. A solution of 700 uL of 2:1 MeOH/HCl (concentrated) was added and heated for 15 minutes at 120° C. After heating, 1 mL of 2 M sodium acetate buffer was added to the reaction solution followed by purification by semi-prep HPLC using a Phenomenex Prodigy ODS-prep C18 column (10 μm 250×10 mm) eluted with 35% acetonitrile/65% 60 mM triethylamine-phosphate buffer (v/v) pH 7.2 at a flow rate of 5 mL/minute for 2 minutes, then the flow was increased to 15 mL/minute for the remainder of the separation. The product, 2-(3-¹⁸F-fluoro-4-methylamino-phenyl)-benzothiazol-6-ol, eluted at ˜15 minutes in a volume of about 16 mL. The fraction containing 2-(3-¹⁸F-fluoro-4-methylamino-phenyl)-benzothiazol-6-ol was diluted with 50 mL of water and eluted through a Waters C18 SepPak Plus cartridge. The SepPak cartridge was then washed with 10 mL of water, and the product was eluted using 1 mL of ethanol (absol.) into a sterile vial. The solution was diluted with 10 mL of sterile normal saline for intravenous injection into animals. The 2-(3-¹⁸F-fluoro-4-methylamino-phenyl)-benzothiazol-6-ol product was obtained in 0.5% (n=4) radiochemical yield at the end of the 120 minute radiosynthesis (not decay corrected) with an average specific activity of 1000 Ci/mmol. The radiochemical and chemical purities of 2-(3-¹⁸F-fluoro-4-methylamino-phenyl)-benzothiazol-6-ol were assessed by radio-HPLC with UV detection at 350 nm using a Phenomenex Prodigy ODS (3) C18 column (5 μm, 250×4.6 mm) eluted with 40% acetonitrile/60% 60 mM triethylamine-phosphate buffer (v/v) pH 7.2. 2-(3-¹⁸F-Fluoro-4-methylamino-phenyl)-benzothiazol-6-ol had a retention time of ˜11 minutes at a flow rate of 2 mL/min (k′=5.5). The radiochemical purity was >99%, and the chemical purity was >90%. The radiochemical identity of 2-(3-¹⁸F-Fluoro-4-methylamino-phenyl)-benzothiazol-6-ol was confirmed by reverse phase radio-HPLC utilizing a quality control sample of the final radiochemical product co-injected with a authentic (cold) standard.

### Example 4

### 2-[4-(3-¹⁸F-Fluoro-propylamino)-phenyl]-benzothiazol-6-ol was synthesized according to Scheme IV

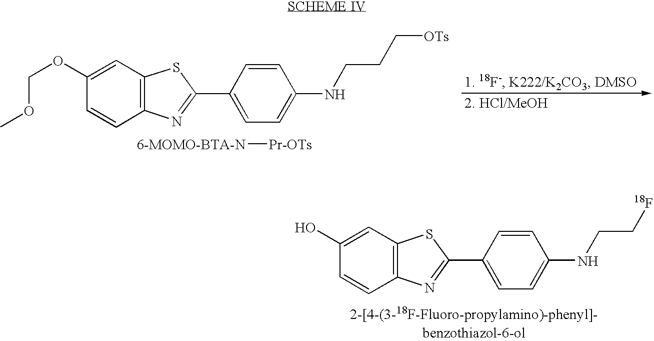

A cyclotron target containing 0.35 mL of 95% [O-18]-enriched water was irradiated with 11 MeV protons at 20 μA of beam current for 60 minutes, and the contents were transferred to a 5 mL reaction vial containing Kryptofix 222 (22.3 mg) and K₂CO₃ (7.9 mg) in acetonitrile (57 μL). The solution was evaporated to dryness three times at 110° C. under a stream of argon following the addition of 1 mL aliquots of acetonitrile. To the dried [F-18]fluoride was added 3 mg of 6-MOMO-BTA-N-Pr-Ots in 1 mL DMSO, and the reaction vial was sealed and heated to 85° C. for 30 minutes. To the reaction vial, 0.5 mL of MeOH/HCl (concentrated) (2/1 v/v) was added, and the vial was heated at 120° C. for 10 minutes. After heating, 0.3 mL of 2 M sodium acetate buffer was added to the reaction solution followed by purification by semi-prep HPLC using a Phenomenex Prodigy ODS-prep C18 column (10 μm 250×10 mm) eluted with 40% acetonitrile/60% 60 mM triethylamine-phosphate buffer (v/v) pH 7.2 at a flow rate of 5 mL/minute for 15 minutes, then the flow was increased to 8 mL/minute for the remainder of the separation. The product, [F-18]6-HO-BTA-N-PrF, eluted at ˜20 minutes in a volume of about 16 mL. The fraction containing[F-18]6-HO-BTA-N-PrF was diluted with 50 mL of water and eluted through a Waters C18 SepPak Plus cartridge. The SepPak cartridge was then washed with 10 mL of water, and the product was eluted using 1 mL of ethanol (absol.) into a sterile vial. The solution was diluted with 10 mL of sterile normal saline for intravenous injection into animals. The [F-18]6-HO-BTA-N-PrF product was obtained in 8±4% (n=8) radiochemical yield at the end of the 120 minute radiosynthesis (not decay corrected) with an average specific activity of 1500 Ci/mmol. The radiochemical and chemical purities of [F-18]6-HO-BTA-N-PrF were assessed by radio-HPLC with UV detection at 350 nm using a Phenomenex Prodigy ODS (3) C18 column (5 μm, 250×4.6 mm) eluted with 40% acetonitrile/60% 60 mM triethylamine-phosphate buffer (v/v) pH 7.2. [F-18]6-HO-BTA-N-PrF had a retention time of ˜12 minutes at a flow rate of 2 mL/minute (k′=6.1). The radiochemical purity was >99%, and the chemical purity was >90%. The radiochemical identity of [F-18]6-HO-BTA-N-PrF was confirmed by reverse phase radio-HPLC utilizing a quality control sample of the final radiochemical product co-injected with a authentic (cold) standard.

### Example 5

### Synthesis of 2-(3′-iodo-4′-aminophenyl)-6-hydroxy benzothiazole

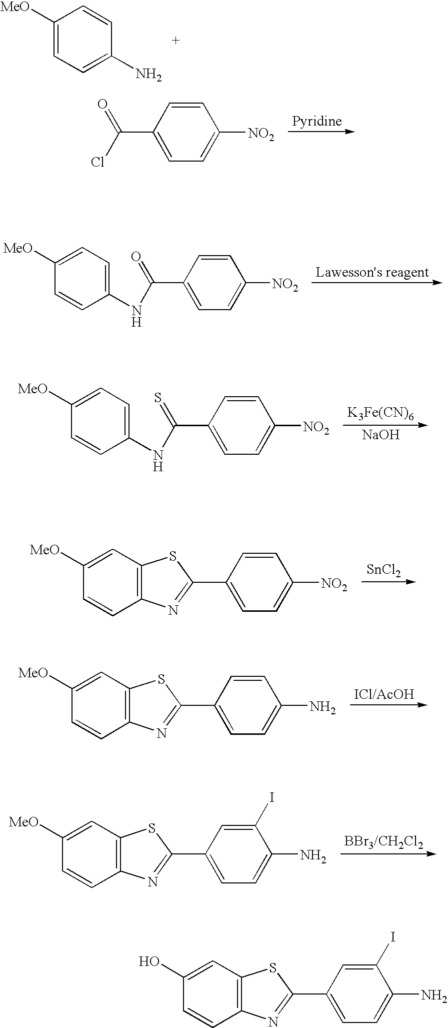

### Preparation of 4-Methoxy-4′-nitrobenzanilide

p-Anisidine (1.0 g, 8.1 mmol) was dissolved in anhydrous pyridine (15 ml), 4-nitrobenzoyl chloride (1.5 g, 8.1 mmol) was added. The reaction mixture was allowed to stand at room temperature for 16 hrs. The reaction mixture was poured into water and the precipitate was collected with filtrate under vacuum pressure and washed with 5% sodium bicarbonate(2×10 ml). The product was used in the next step without further purification. ¹HNMR (300 MHz, DMSO-d₆) δ: 10.46 (s, 1H, NH), 8.37 (d, J=5.5 Hz, 2H, H-3′,5′), 8.17 (d, J=6.3 Hz, 2H, H-2′,6′), 7.48 (d, J=6.6 Hz, 2H), 6.97 (d, J=6.5 Hz, 2H), 3.75 (s, 3H, MeO).

### Preparation of 4-Methoxy-4′-nitrothiobenzanilide

A mixture of 4-methoxy-4′-nitrothiobenzaniline (1.0 g, 3.7 mmol) and Lawesson's reagent (0.89 g, 2.2 mmol, 0.6 equiv.) in chlorobenzene (15 mL) was heated to reflux for 4 hrs. The solvent was evaporated and the residue was purified with flush column (hexane:ethyl acetate=4:1) to give 820 mg (77.4%) of the product as orange color solid. ¹HNMR (300 MHz, DMSO-d₆) δ: 8.29 (d, 2H, H-3′,5′), 8.00 (d, J=8.5 Hz, 2H, H-2′,6′), 7.76 (d, 2H), 7.03 (d, J=8.4 Hz, 2H), 3.808.37 (d, J=5.5 Hz, 2H, H-3′,5′), 8.17 (d, J=6.3 Hz, 2H, H-2′,6′), 7.48 (d, J=6.6 Hz, 2H), 6.97 (d, J=6.5 Hz, 2H), 3.75 (s, 3H, MeO). (s, 3H, MeO).

### Preparation of 6-Methoxy-2-(4-nitrophenyl)benzothiazole

4-Methoxy-4′-nitrothiobenzanilides (0.5 g, 1.74 mmol) was wetted with a little ethanol(˜0.5 mL), and 30% aqueous sodium hydroxide solution (556 mg 13.9 mmol. 8 equiv.) was added. The mixture was diluted with water to provide a final solution/suspension of 10% aqueous sodium hydroxide. Aliquots of this mixture were added at 1 min intervals to a stirred solution of potassium ferricyanide (2.29 g, 6.9 mmol, 4 equiv.) in water (5 mL) at 80-90° C. The reaction mixture was heated for a further 0.5 h and then allowed to cool. The participate was collected by filtration under vacuum pressure and washed with water, purified with flush column (hexane:ethyl acetate=4:1) to give 130 mg (26%) of the product. ¹HNMR (300 MHz, Acetone-d₆) δ: 8.45 (m, 4H), 8.07 (d, J=8.5 Hz, 1H, H-4), 7.69 (s, 1H, H-7), 7.22 (d, J=9.0 Hz, 1H, H-5), 3.90 (s, 3H, MeO)

### Preparation of 6-Methoxy-2-(4-aminophenyl)benzothiazole

A mixture of the 6-methoxy-2-(4-nitrophenyl)benzothiazoles (22 mg, 0.077 mmol) and tin(II) chloride (132 mg, 0.45 mmol) in boiling ethanol was stirred under nitrogen for 4 hrs. Ethanol was evaporated and the residue was dissolved in ethyl acetate (10 mL), washed with 1 N sodium hydroxide (2 mL) and water (5 mL), and dried over MgSO₄. Evaporation of the solvent gave 19 mg (97%) of the product as yellow solid.

### Preparation of 2-(3′-Iodo-4′-aminophenyl)-6-methoxybenzothiazole

To a solution of 2-(4′-aminophenyl)-6-methoxy benzothiazole (22 mg, 0.09 mmol) in glacial acetic acid (2.0 mL) was injected 1 M iodochloride solution in CH₂Cl₂ (0.10 mL, 0.10 mmol, 1.2 eq.) under N₂ atmosphere. The reaction mixture was stirred at room temperature for 16 hr. The glacial acetic acid was removed under reduced pressure and the residue was dissolved in CH₂Cl₂. After neutralizing the solution with NaHCO₃, the aqueous layer was separated and extracted with CH₂Cl₂. The organic layers were combined and dried over MgSO₄. Following the evaporation of the solvent, the residue was purified by preparative TLC(Hexanes:ethyl acetate=6:1) to give 2-(4′-amino-3′-iodophenyl)-6-methoxy benzothiazole (25 mg, 76%) as brown solid. ¹HNMR (300 MHz, CDCl₃) δ (ppm): 8.35 (d, J=2.0 Hz, 1H), 7.87 (dd, J₁=2.0 Hz, J2=9.0 Hz, 1H), 7.31 (d, J=2.2 Hz, 1H), 7.04 (dd, J₁=2.2 Hz, J₂=9.0 Hz, 1H), 6.76 (d, J=9.0 Hz, 1H), 3.87 (s, 3H).

### Preparation of 2-(3′-Iodo-4′-aminophenyl)-6-hydroxybenzothiazole

To a solution of 2-(4′-Amino-3′-iodophenyl)-6-methoxy benzothiazole (5) (8.0 mg, 0.02 mmol) in CH₂Cl₂ (2.0 mL) was injected 1 M BBr₃ solution in CH₂Cl₂ (0.20 ml, 0.20 mmol) under N₂ atmosphere. The reaction mixture was stirred at room temperature for 18 hrs. After the reaction was quenched with water, the mixture was neutralized with NaHCO₃. The aqueous layer was extracted with ethyl acetate(3×3 mL). The organic layers were combined and dried over MgSO₄. The solvent was then evaporated under reduced pressure and the residue was purified by preparative TLC (Hexanes:ethyl acetate=7:3) to give 2-(3′-iodo-4′-aminophenyl)-6-hydroxybenzothiazole (4.5 mg, 58%) as a brown solid. ¹HNMR (300 MHz, acetone-d₆) δ (ppm): 8.69 (s, 1H), 8.34 (d, J=2.0 Hz, 1H), 7.77 (dd, J₁=2.0 Hz, J₂=8.4 Hz, 1H), 7.76 (d, J=8.8 Hz, 1H), 7.40 (d, J=2.4 Hz, 1H), 7.02 (dd, J₁=2.5 Hz, J₂=8.8 Hz, 1H), 6.94 (d, J=8.5 Hz, 1H), 5.47 (br., 2H). HRMS m/z 367.9483 (M⁺ calcd for C₁₃H₉N₂OSI 367.9480).

### Example 6

### Synthesis of 2-(3′-iodo-4′-methylaminophenyl)-6-hydroxybenzothiazole

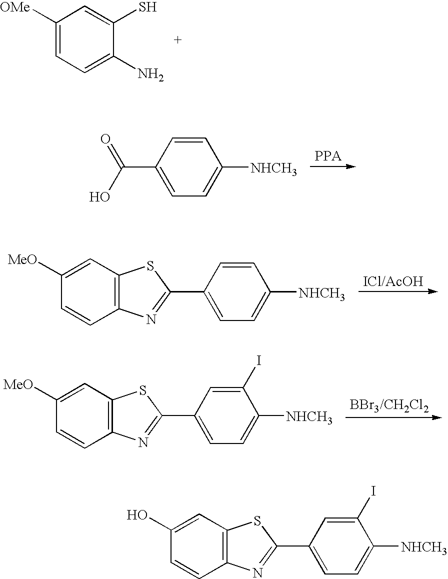

### Preparation of 6-Methoxy-2-(4-methylaminophenyl)benzothiazole

A mixture of 4-methylaminobenzoic acid (11.5 g, 76.2 mmol) and 5-methoxy-2-aminothiophenol (12.5, g, 80 mmol) was heated in PPA (˜30 g) to 170° C. under N₂ atmosphere for 1.5 hr. The reaction mixture was then cooled to room temperature and poured into 10% K₂CO₃ solution. The precipitate was filtered under reduced pressure. The crude product was re-crystallized twice from acetone/water and THF/water followed by the treatment with active with carbon to give 4.6 g (21%) of 6-Methoxy-2-(4-methylaminophenyl)benzothiazole as a yellow solid. ¹HNMR (300 MHz, acetone-d₆) δ: 7.84 (d, J=8.7 Hz, 2H, H-2′ 6′), 7.78 (dd, J₁=8.8 Hz, J₂=1.3 Hz, 1H, H-4), 7.52 (d, J=2.4 Hz, 1H, H-7), 7.05 (dd, J₁=8.8 Hz, J₂=2.4 Hz, H-5), 6.70 (d, J=7.6 Hz, 2H, H-3′ 5′), 5.62 (s, 1H, NH), 3.88 (s, 3H, OCH₃), 2.85 (d, J=6.2 Hz, 3H, NCH₃)

### Preparation of 2-(3′-Iodo-4′-methylaminophenyl)-6-methoxy benzothiazole

To a solution of 2-(4′-Methylaminophenyl)-6-methoxy benzothiazole (20 mg, 0.074 mmol) dissolved in glacial acetic acid (2 mL) was added Icl (90 μL, 0.15 mmol, 1.2 eq, 1M in CH₂Cl₂) under N₂. The reaction was allowed to stir at room temperature for 18 hr. The glacial acetic acid was then removed under reduced pressure. The residue was dissolved in CH₂Cl₂ and neutralized with NaHCO₃. The aqueous layer was extracted with CH₂Cl₂ and the organic layers were combined, dried over MgSO₄ and evaporated. The residue was purified with preparative TLC (Hexane:EA=2:1) to give 2-(4′-methylamino-3′-iodophenyl)-6-methoxy benzothiazole (8 mg, 27%) as brown solid. ¹HNMR (300 MHz, CDCl₃) δ(ppm): 8.39 (d, J=2.0 Hz, 1H), 7.88 (d, J=9.0 Hz, 1H), 7.33 (d, J=2.2 Hz, 1H), 7.06 (dd, J₁=2.2 Hz, J₂=9.0 Hz, 1H), 6.58 (d, J=9.0 Hz, 1H), 3.89 (s, 3H, OCH₃).

### Preparation of 2-(3′-Iodo-4′-methylamino-phenyl)-6-hydroxy benzothiazole

To a solution of 2-(4′-methylamino-3′-iodophenyl)-6-methoxy benzothiazole (12 mg, 0.03 mmol) dissolved in CH₂Cl₂(4 mL) was added BBr₃ (400 μl, 0.4 mmol, 1M in CH₂Cl₂) under N₂. The reaction was allowed to stir at room temperature for 18 hr. Water was then added to quench the reaction and the solution was neutralized with NaHCO₃, extracted with ethyl acetate (3×5 mL). The organic layers were combined, dried over MgSO₄ and evaporated. The residue was purified with preparative TLC (Hexane: EA=7:3) to give 2-(4′-methylamino-3′-iodophenyl)-6-hydroxy benzothiazole (5 mg, 43%) as brown solid. ¹HNMR (300 MHz, CDCl₃) δ(ppm): 8.37 (d, H=2.0 Hz, 1H), 7.88 (dd, J₁=2.0 Hz, J₂=8.4 Hz, 1H), 7.83 (d, J=8.8 Hz, 1H), 7.28 (d, J=2.4 Hz, 1H), 6.96 (dd, J₁=2.5 Hz, J₂=8.8 Hz, 1H), 6.58 (d, J=8.5 Hz, 1H), 2.96 (s, 3H, CH₃).

### Example 7

### Radiosynthesis of [¹²⁵I]6-OH-BTA-0-3′-I

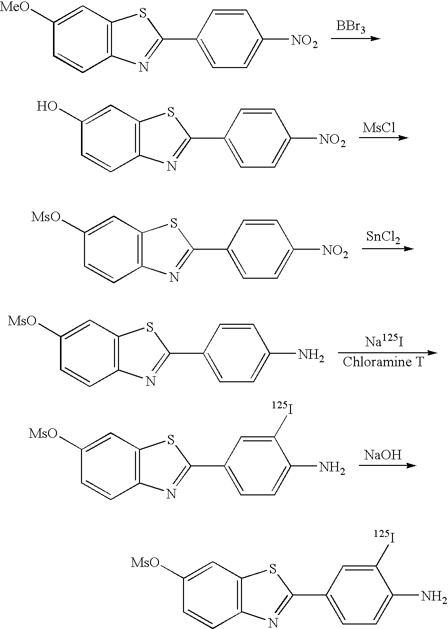

### Preparation of 2-(4′-Nitrophenyl)-6-hydroxybenzothiazole

To a suspension of 2-(4′-nitrophenyl)-6-methoxy benzothiazole (400 mg, 1.5 mmol) in CH₂Cl₂ (10 mL) was added BBr₃ (1M in CH₂Cl₂, 10 mL, 10 mmol). The reaction mixture was stirred at room temperature for 24 hr. The reaction was then quenched with water, and extracted with ethyl acetate (3×20 mL). The organic layers were combined and washed with water, dried over MgSO₄, and evaporated. The residue was purified by flash chromatography (silica gel, hexanes:ethyl acetate=1:1) to give the product as a yellow solid (210 mg, 55%). ¹HNMR (300 MHz, Acetone-d₆) δ (ppm): 9.02 (s, OH), 8.41 (d, J=9.1 Hz, 1H), 8.33 (d, J=9.1 Hz, 1H), 7.96 (d, J=8.6 Hz, 1H), 7.53 (d, J=2.4 Hz, 1H), 7.15 (dd, J1=8.6 Hz, J2=2.4 Hz, 1H).

### Preparation of 2-(4′-Nitrophenyl)-6-methylsulfoxy benzothiazole

To a solution of 2-(4′-nitrophenyl)-6-hydroxy benzothiazole (50 mg, 0.18 mmol) dissolved in acetone (7 mL, anhydrous) was added K₂CO₃ (100 mg, 0.72 mmol, powdered) and MsCl (200 ul). After stirring for 2 hrs, the reaction mixture was filtered. The filtrate was concentrated and the residue was purified by flash column (silica gel, hexane:ethyl acetate=4:1) to give 2-(4-nitrophenyl)-6-methylsulfoxy benzothiazole (44 mg, 68%) as pale yellow solid. ¹HNMR (300 MHz, acetone-d₆) δ (ppm): 8.50-8.40 (m, 4H), 8.29 (d, J=2.3 Hz, 1H), 8.23 (d, J=8.9 Hz, 1H), 7.61 (dd, J₁=2.3 Hz, J₂=8.9 Hz, 1H).

### Preparation of 2-(4′-Aminophenyl)-6-methylsulfoxy benzothiazole

To a solution of 2-(4′-nitrophenyl)-6-methylsulfoxy benzothiazole (35 mg, 0.10 mmol) dissolved in ethanol (10 mL) was added SnCl₂.2H₂O (50 mg). The reaction mixture was heated to reflux for 1.5 hr. The solvent was then removed under reduced pressure. The residue was dissolved in ethyl acetate (10 mL), washed with 1N NaOH, water, dried over MgSO₄. Evaporation of the solvent afforded 2-(4′-aminophenyl)-6-methylsulfoxy benzothiazole (21 mg, 65%) as pale brown solid. ¹HNMR (300 MHz, CDCl₃) δ (ppm): 8.02 (d, J=6.2 Hz, 1H), 7.92 (d, J=8.7 Hz, 2H), 7.84 (d, J=2.4 Hz, 1H), 7.38 (dd, J₁=2.4 Hz, J₂=6.2 Hz, 1H), 6.78 (d, J=8.7 Hz, 2H), 2.21 (s, 3H, CH₃).

### Example 8

### Radiosynthesis of [¹²⁵I]6-OH-BTA-1-3′-I

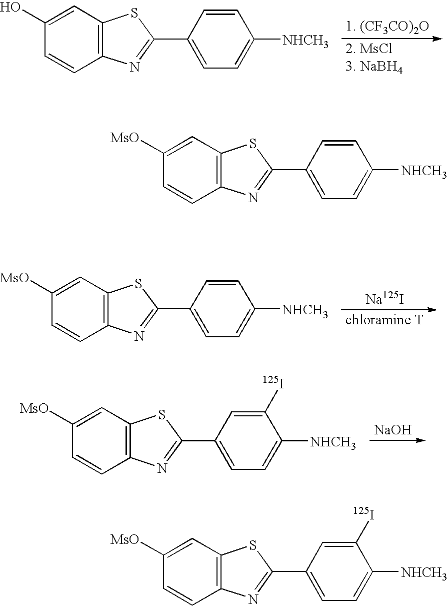

To a solution of 2-(4′-methylaminophenyl)-6-hydroxy benzothiazole (300 mg, 1.17 mmol) dissolved in CH₂Cl₂ (20 mL) was added Et₃N (2 mL) and trifluoroacetic acid (1.5 mL). The reaction mixture was stirred at room temperature for 3 h. The solvent was removed under reduced pressure and the residue was dissolved in ethyl acetate (30 mL), washed with NaHCO₃ solution. Brine, water, and dried over MgSO₄. After evaporation of the solvent, the residue was dissolved in acetone (20 ml, pre-dried over K₂CO₃), K₂CO₃ (1.0 g, powered) was added followed by MsCl (400 mg, 3.49 mmol). The reaction mixture was stirred at room temperature and monitored with TLC □ omog starting material disappeared. The residue was then filtrated. The filtrate was evaporated under reduced pressure. The residue was dissolved in ethyl acetate (30 mL), washed with NaHCO₃ solution. Brine, water, and dried over MgSO₄. After evaporation of the solvent, the residue was dissolved in EtOH and NaBH₄ was added. The reaction mixture was stirred at room temperature for 2 h. The solvent was evaporated and the residue was dissolved in water, extracted with ethyl acetate (20 ml×3), the extracts were combined and dried over MgSO₄. After evaporation of the solvent, the residue was purified with flash column(hexanes/ethyl acetate=8:1) to give the product (184 mg, 47.0%) as brown solid. ¹HNMR (300 MHz, CDCl₃) δ (ppm): 7.94 (d, J=8.8 Hz, 1H), 7.87 (d, J=8.7 Hz, 2H), 7.77 (d, J=2.3 Hz, 1H), 7.30 (dd, J₁=8.8 Hz, J₂=2.3 Hz, 1H), 6.63 (d, J=8.7 Hz, 2H), 3.16 (s, CH₃), 2.89 (s, NCH₃).

### General Procedures for Radiolabelling:

To a solution of 2-(4′-aminophenyl)-6-methanesulfonoxy benzothiazole or 2-(4′-methylaminophenyl)-6-methylsulfoxy benzothiazole (1 mg) in 250 μL acetic acid in a sealed vial was added 40 μL of chloramines T solution (28 mg dissolved in 500 μL acetic acid) followed by 27 μL (ca. 5 mCi) of sodium [¹²⁵I]iodide (specific activity 2,175 Ci/mmol). The reaction mixture was stirred at r.t. for 2.5 hrs and quenched with saturated sodium hydrogensulfite solution. After dilution with 20 ml of water, the reaction mixture was loaded onto C8 Plus SepPak and eluted with 2 ml methanol. For deprotection of the methanesulfonyl group, 0.5 ml of 1 M NaOH was added to the eluted solution of radioiodinated intermediate. The mixture was heated at 50° C. for 2 hours. After being quenched by 500 μL of 1 M acetic acid, the reaction mixture was diluted with 40 mL of water and loaded onto a C8 Plus SepPak. The radioiodinated product, having a radioactivity of ca. 3 mCi, was eluted off the SepPak with 2 mL of methanol. The solution was condensed by a nitrogen stream to 300 μL and the crude product was purified by HPLC on a Phenomenex ODS column (MeCN/TEA buffer, 35:65, pH 7.5, flow rate 0.5 mL/min up to 4 min, 1.0 mL/min at 4-6 min, and 2.0 mL/min after 6 min, retention time 23.6). The collected fractions were loaded onto a C8 Plus SepPak. Elution with 1 mL of ethanol gave ca. 1 mCi of the final radioiodinated product.

### BIOLOGICAL EXAMPLE

### Example 9

### Imaging of Tissues from AL Amyloidosis Subject

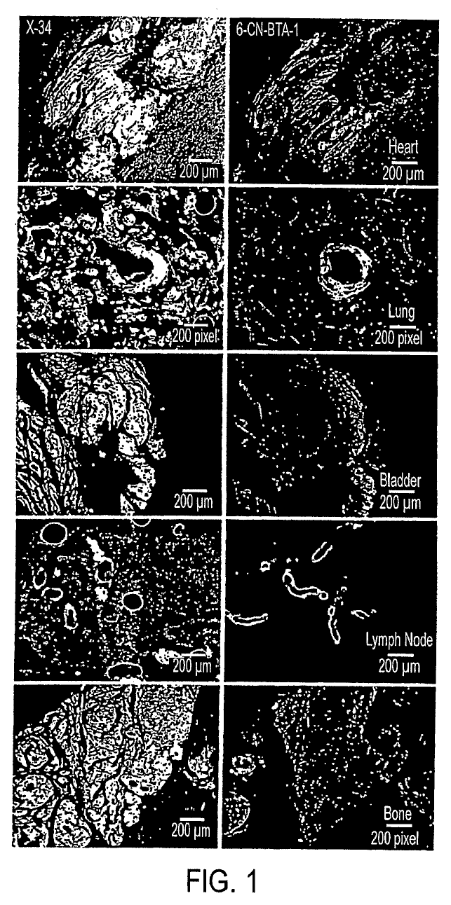

Paraffin sections of heart, lung, bladder, lymph node and bone from a subject with AL amyloidosis were deparaffinized in xylene and stained with 100 nM X-34 [Styren et al. J Histochem Cytochem 48:1223-1232 (2000)] in 20% ethanol/80% 150 mM Tris Buffer (pH 7.4) or 100 nM 2-(4′-methylaminophenyl)-6-cyanobenzothiazole (6-CN-BTA-1) [Mathis et al. J Med Chem 46:2740-2754 (2003)] in PBS (pH 7.4) for 60 min followed by a brief, 5 second wash in water followed by coverslipping and viewing with an UV filter set (FIG. 1).

Other embodiments of the invention will be apparent to those skilled in the art from consideration of the specification and practice of the invention disclosed herein. It is intended that the specification be considered as exemplary only, with the true scope and spirit of the invention being indicated by the following claims.

As used herein and in the following claims, singular articles such as “a”, “an”, and “one” are intended to refer to singular or plural.

What is claimed is:

1. An in vivo method for detecting in a subject at least one amyloid deposit comprising at least one amyloidogenic protein, comprising the steps of:
(a) administering to a subject suffering from a disease associated with amyloidosis, a detectable quantity of a pharmaceutical composition comprising at least one compound of formula I and a pharmaceutically acceptable carrier, 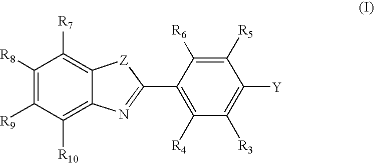
wherein
(i) Z is S, NR′, O or C(R′)₂, such that when Z is C(R′)₂, the tautomeric form of the heterocyclic ring may form an indole: 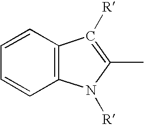
wherein R′ is H or a lower alkyl group,
(ii) Y is NR¹R², OR², or SR²,
(iii) R¹ is selected from the group consisting of H, a lower alkyl group, (CH₂)ₙOR′ (wherein n=1, 2, or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3, or 4 and Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′ and COOR′, where R′ is H or a lower alkyl group);
(iv) R² is selected from the group consisting of H, a lower alkyl group, (CH₂)ₙOR′ (wherein n=1, 2, or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3, or 4 and Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′ and COOR′, where R′ is H or a lower alkyl group);
(v) R³ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(vi) R⁴ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(vii) R⁵ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(viii) R⁶ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(ix) R⁷ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(x) R⁸ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(xi) R⁹ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(xii) R¹⁰ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
alternatively, one of R³-R¹⁰ may be a chelating group, with or without a chelated metal group, said chelating group being of the form W-L or V-W-L, wherein V is selected from the group consisting of —COO—, —CO—, —CH₂O— and —CH₂NH—; W is —(CH₂)ₙ where n=0, 1, 2, 3, 4, or 5, and L is: 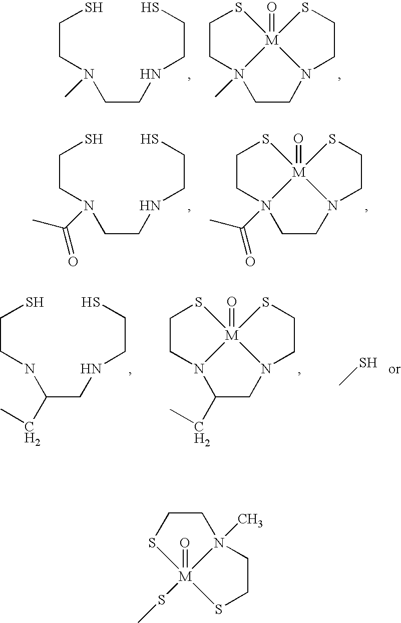
wherein M is selected from the group consisting of Tc and Re and radiolabelled derivatives and pharmaceutically acceptable salts thereof, where at least one of the substituent moieties comprises a detectable label; and
(b) detecting the binding of the compound to an amyloid deposit comprising at least one amyloidogenic protein, wherein the amyloidogenic protein is selected from the group consisting of AL, AH, ATTR, Aβ2M, AA, AApoAI, AApoAII, AGel, ALys, AFib, ACys, ABri, ADan, APrP, ACal, AlAPP, AANF, APro, AIns, AMed, AKer, A(tbn), and ALac.

2. An in vitro method for detecting in a subject at least one amyloid deposit comprising at least one amyloidogenic protein, comprising the steps of
(a) obtaining a fresh or frozen tissue specimen and incubating a section of the tissue or a homogenate of the tissue with a radioactively labeled thioflavin derivative of formula (I):
formula I and a pharmaceutically acceptable carrier, 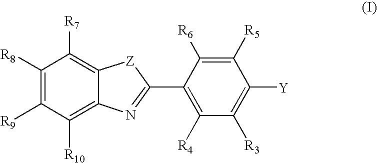
wherein
(i) Z is S, NR′, O or C(R′)₂, such that when Z is C(R′)₂, the tautomeric form of the heterocyclic ring may form an indole: 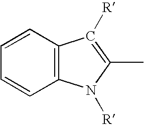
wherein R′ is H or a lower alkyl group,
(ii) Y is NR¹R², OR², or SR²,
(iii) R¹ is selected from the group consisting of H, a lower alkyl group, (CH₂)ₙOR′ (wherein n=1, 2, or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3, or 4 and Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′ and COOR′, where R′ is H or a lower alkyl group);
(iv) R² is selected from the group consisting of H, a lower alkyl group, (CH₂)ₙOR′ (wherein n=1, 2, or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3, or 4 and Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′ and COOR′, where R′ is H or a lower alkyl group);
(v) R³ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(vi) R⁴ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(vii) R⁵ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(viii) R⁶ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(ix) R⁷ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(x) R⁸ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(xi) R⁹ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(xii) R¹⁰ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
alternatively, one of R³-R¹⁰ may be a chelating group, with or without a chelated metal group, said chelating group being of the form W-L or V-W-L, wherein V is selected from the group consisting of —COO—, —CO—, —CH₂O— and —CH₂NH—; W is —(CH₂)ₙ where n=0, 1, 2, 3, 4, or 5, and L is: 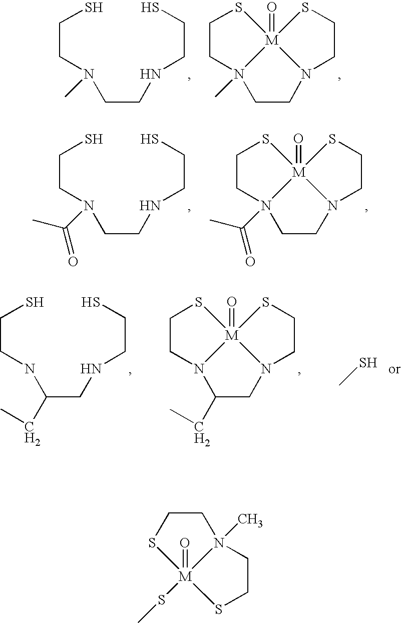
wherein M is selected from the group consisting of Tc and Re and radiolabelled derivatives and pharmaceutically acceptable salts thereof,
where at least one of the substituent moieties comprises a detectable label;
(b) then separating bound and free radiolabel by washing the tissue section or filtering and washing the tissue homogenate; and
(c) detecting the binding of the compound to an amyloid deposit comprising at least one amyloidogenic protein, wherein the amyloidogenic protein is selected from the group consisting of AL, AH, ATTR, Aβ2M, AA, AApoAI, AApoAII, AGel, ALys, AFib, ACys, ABri, ADan, APrP, ACal, AlAPP, AANF, APro, AIns, AMed, AKer, A(tbn), and ALac.

3. The method of claim 1 or 2, wherein the at least one amyloidogenic protein is derived from at least one protein precursor selected from the group consisting of immunoglobulin light chain, immunoglobulin heavy chain, transthyretin, β2-microglobulin, (Apo)serum AA, Apolipoprotien AI, Apolipoprotein AII, gelsolin, lysozyme, fibrinogen α-chain, cystatin C, ABriPP, ADanPP, prion protein, (Pro)calcitonin, islet amyloid polypeptide, atrial natriuretic factor, prolactin, insulin, lactadherin, kerato-epithelin, Pindborg tumor associated precursor protein (tbn) and lactoferrin.

4. The method of claim 1, wherein the subject is suffering from a disease associated with systemic amyloidosis.

5. The method of claim 3, wherein the at least one amyloid deposit is located in a mesodermal tissue of the subject.

6. The method of claim 4, wherein the tissue is selected from the group consisting of peripheral nerve, skin, tongue, joint, heart or liver.

7. The method of claim 3, wherein the at least one amyloid deposit is located in a parenchymatous organ.

8. The method of claim 6, wherein the organ is selected from the group consisting of spleen, kidney, liver and adrenal.

9. The method of claim 3, wherein the disease associated with systemic amyloidosis is selected from the group consisting of multiple myeloma, macroglobulinemia, lymphoma, chronic inflammatory disease, rheumatoird arthritis, infectious disease, dermatomyositis, scleroderma, regional enteritis, ulcerative colitis, tuberculosis, chronic osteomyelitis, bronchiectasis, skin abscess, lung abscess, cancer, Hodgkin's disease, heredofamilial amyloidosis, familial Mediterranean fever, familial dementia and familial amyloid polyneuropathy.

10. The method of claim 8, where said skin or lung abscess results from subcutaneous heroin use.

11. The method of claim 1, where the disease is cerebral amyloid angiopathy.

12. The method of claim 1, wherein the detecting is selected from the group consisting of gamma imaging, magnetic resonance imaging and magnetic resonance spectroscopy.

13. The method of claim 1, wherein the detecting is done by gamma imaging, and the gamma imaging is either PET or SPECT.

14. The method of claim 1, wherein the pharmaceutical composition is administered by intravenous injection.

15. The method of claim 1, wherein the subject is receiving hemodialysis for chronic renal failure.

16. The method of claim 1, wherein the subject is suffering from a disease associated with localized amyloidosis.

17. The method of claim 15, wherein the at least one amyloid deposit is located in a tissue selected from the group consisting of tenosynovium, joints, aortic, thyroid, islets of langerhans, aging pituitary, latrogenic, cardiac atria, and cornea.

18. The method of claim 15, wherein the at least one amyloid deposit is located in the pancreas.

19. The method of claim 15, wherein the disease associated with localized amyloidosis is selected from the group consisting of primary myeloma, familial dementia, spongioform encephalopathies, c-cell thyroid tumor, insulinoma, prolactinoma and pindborg tumor.

20. The method of claim 1, wherein the compound of Formula (I) comprises a compound of formula (II): 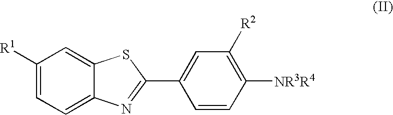
or a radiolabeled derivative, pharmaceutically acceptable salt, hydrate, solvate or prodrug of the compound, wherein:
R¹ is hydrogen, —OH, —NO₂, —CN, —COOR, —OCH₂OR, C₁-C₆ alkyl, C₂-C₆ alkenyl, C₂-C₆ alkynyl, C₁-C₆ alkoxy or halo;
R is C₁-C₆ alkyl;
R² is hydrogen or halo;
R³ is hydrogen, C₁-C₆ alkyl, C₂-C₆ alkenyl or C₂-C₆ alkynyl; and
R⁴ is hydrogen, C₁-C₆ alkyl, C₂-C₆ alkenyl or C₂-C₆ alkynyl, wherein the alkyl, alkenyl or alkynyl comprises a radioactive carbon or is substituted with a radioactive halo when R² is hydrogen or a non-radioactive halo;
provided that when R¹ is hydrogen or —OH, R² is hydrogen and R⁴ is —¹¹CH₃, then R³ is C₂-C₆ alkyl, C₂-C₆ alkenyl or C₂-C₆ alkynyl; and
further provided that when R¹ is hydrogen, R² hydrogen and R⁴ is —(CH₂)₃¹⁸F, then R³ is C₂-C₆ alkyl, C₂-C₆ alkenyl or C₂-C₆ alkynyl, where at least one of the substituent moieties comprises a detectable label.

21. The method of claim 1, where the amyloid imaging agent of formula (I) is selected from the group consisting of structures 1-45 or a radiolabeled derivative thereof, wherein the compound comprises at least one detectable label:

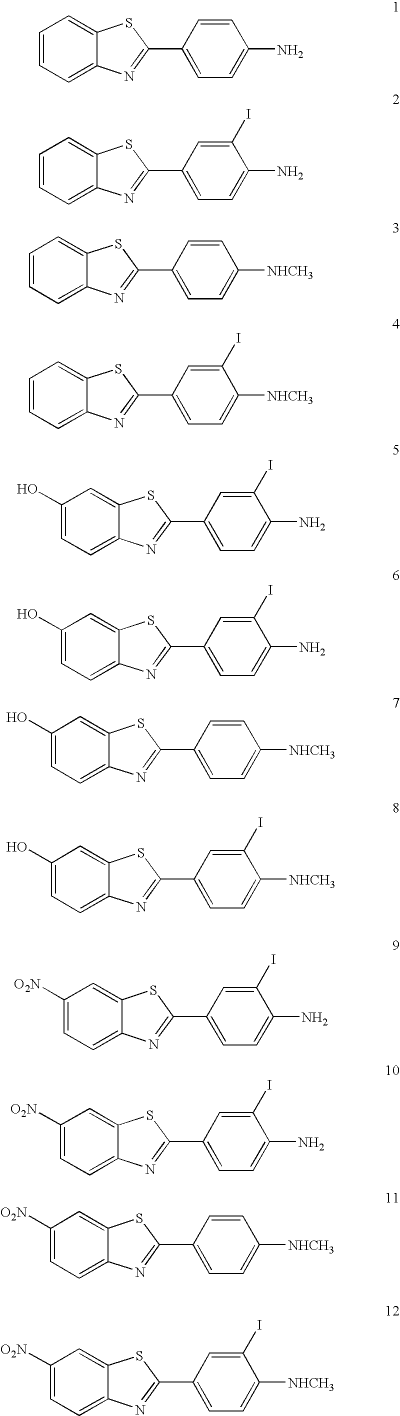

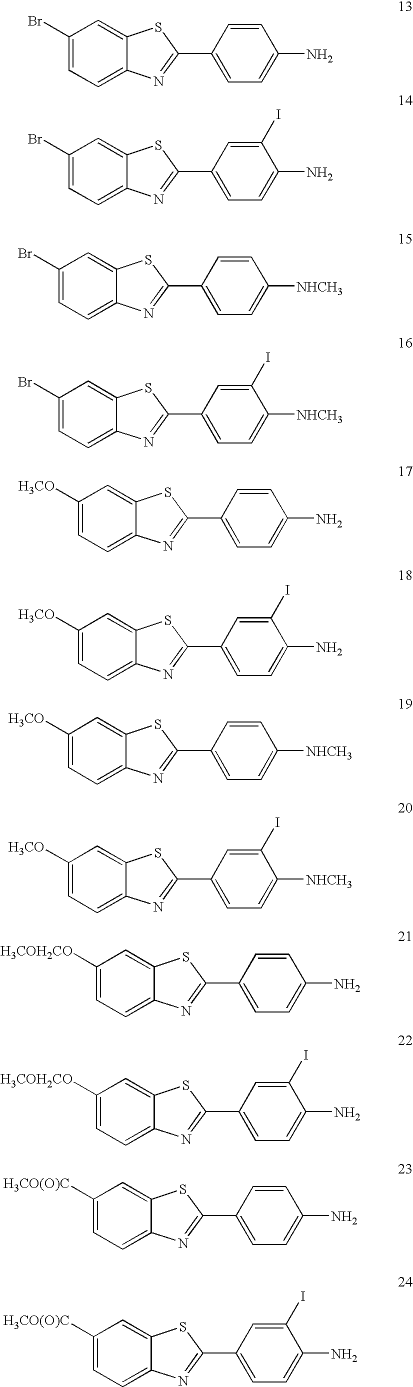

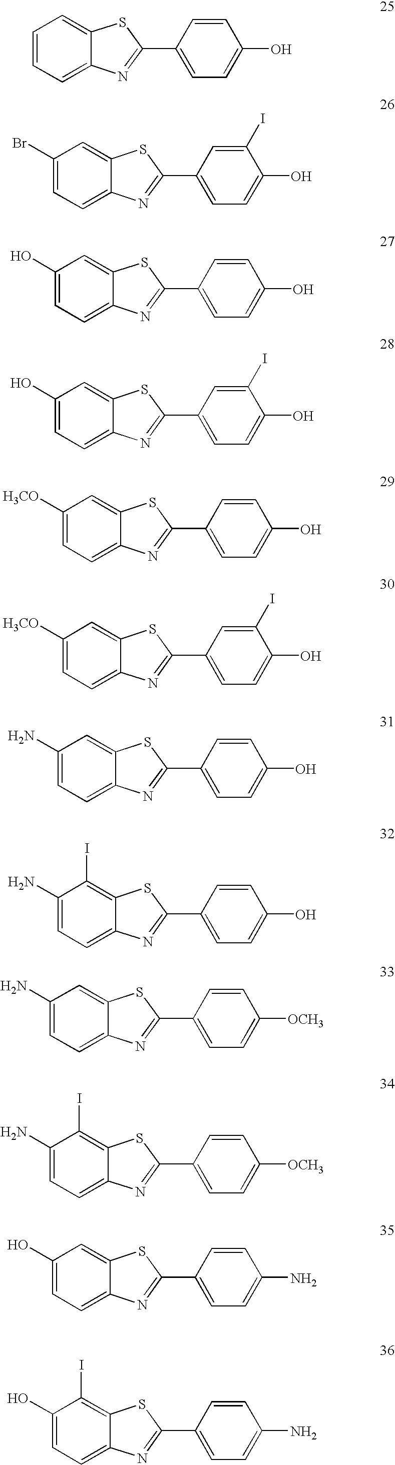

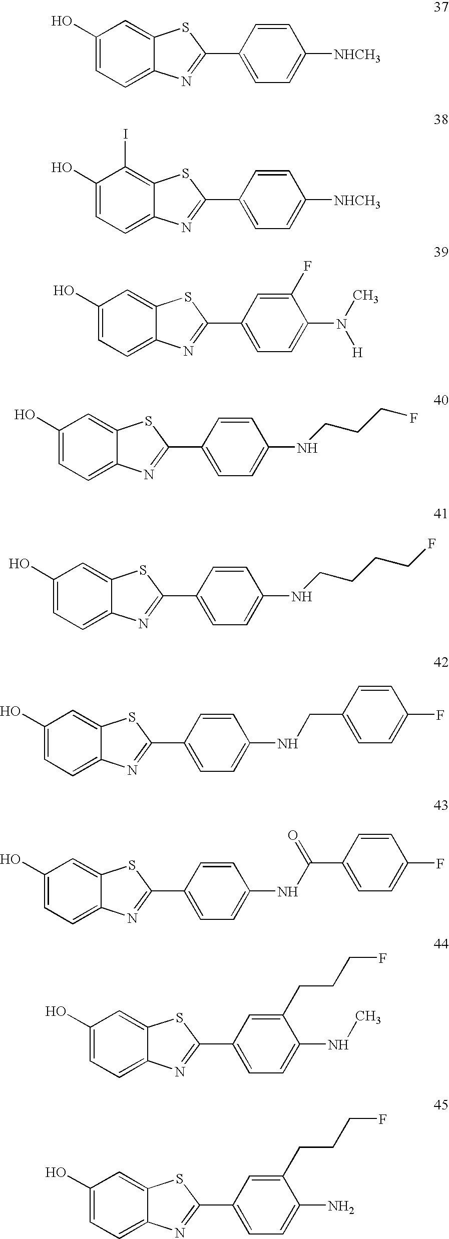

22. An in vivo method for detecting in a subject at least one amyloid deposit comprising at least one amyloidogenic protein, comprising the steps of:
(a) administering to a subject suffering from a disease associated with amyloidosis, a detectable quantity of a pharmaceutical composition comprising at least one compound of formula I and a pharmaceutically acceptable carrier, 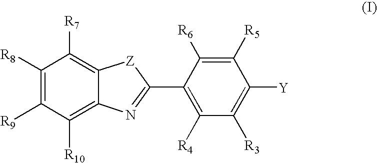
wherein
(i) Z is S, NR′, O or C(R′)₂, such that when Z is C(R′)₂, the tautomeric form of the heterocyclic ring may form an indole: 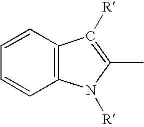
wherein R′ is H or a lower alkyl group,
(ii) Y is NR¹R², OR², or SR²,
(iii) R¹ is selected from the group consisting of H, a lower alkyl group, (CH₂)ₙOR′ (wherein n=1, 2, or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3, or 4 and Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′ and COOR′, where R¹ is H or a lower alkyl group);
(iv) R² is selected from the group consisting of H, a lower alkyl group, (CH₂)ₙOR′ (wherein n=1, 2, or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3, or 4 and Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′ and COOR′, where R′ is H or a lower alkyl group);
(v) R³ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(vi) R⁴ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(vii) R⁵ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(viii) R⁶ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(ix) R⁷ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(x) R⁸ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(xi) R⁹ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(xii) R¹⁰ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
alternatively, one of R³-R¹⁰ may be a chelating group, with or without a chelated metal group, said chelating group being of the form W-L or V-W-L, wherein V is selected from the group consisting of —COO—, —CO—, —CH₂O— and —CH₂NH—; W is —(CH₂)ₙ where n=0, 1, 2, 3, 4, or 5, and L is: 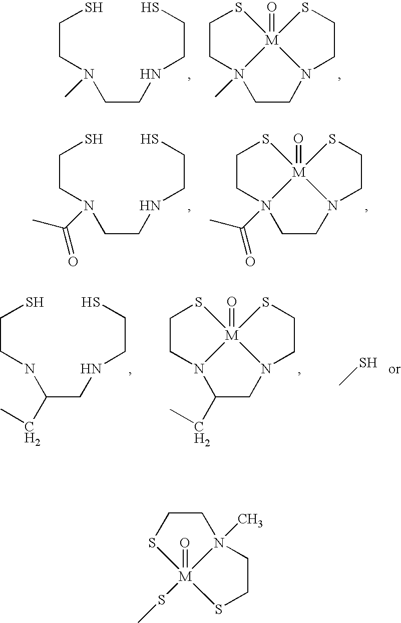
wherein M is selected from the group consisting of Tc and Re and radiolabelled derivatives and pharmaceutically acceptable salts thereof, where at least one of the substituent moieties comprises a detectable label,
whereby the compound binds to the amyloid deposit comprising at least one amyloidogenic protein, wherein the amyloidogenic protein is selected from the group consisting of AL, AH, ATTR, Aβ2M, AA, AApoAI, AApoAII, AGel, ALys, AFib, ACys, ABri, ADan, APrP, ACal, AlAPP, AANF, APro, AIns, AMed, AKer, A(tbn), and ALac;
(b) irradiating the subject and collecting imaging data emitted by the compound; and
(c) processing the imaging data.

23. Use of a compound according to formula (I): 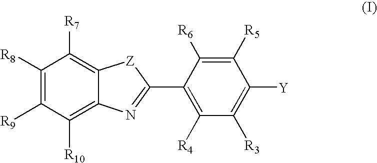
wherein
(i) Z is S, NR′, O or C(R′)₂, such that when Z is C(R′)₂, the tautomeric form of the heterocyclic ring may form an indole: 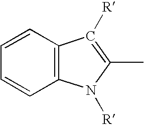
wherein R′ is H or a lower alkyl group,
(ii) Y is NR¹R², OR², or SR²,
(iii) R¹ is selected from the group consisting of H, a lower alkyl group, (CH₂)ₙOR′ (wherein n=1, 2, or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3, or 4 and Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′ and COOR′, where R′ is H or a lower alkyl group);
(iv) R² is selected from the group consisting of H, a lower alkyl group, (CH₂)ₙOR′ (wherein n=1, 2, or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3, or 4 and Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′ and COOR′, where R′ is H or a lower alkyl group);
(v) R³ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(vi) R⁴ is selected from the group consisting of H, F, Cl, Br, I, a lower alkyl group, (CH₂)ₙOR′ (wherein n−1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(vii) R⁵ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(viii) R⁶ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(ix) R⁷ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(x) R⁸ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(xi) R⁹ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(xii) R¹⁰ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
alternatively, one of R³-R¹⁰ may be a chelating group, with or without a chelated metal group, said chelating group being of the form W-L or V-W-L, wherein V is selected from the group consisting of —COO—, —CO—, —CH₂O— and —CH₂NH—; W is —(CH₂)ₙ where n=0, 1, 2, 3, 4, or 5, and L is: 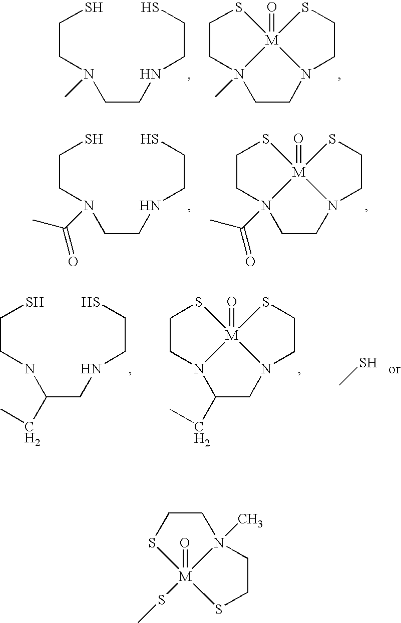
wherein M is selected from the group consisting of Tc and Re and radiolabelled derivatives and pharmaceutically acceptable salts thereof, where at least one of the substituent moieties comprises a detectable label,
for the detection of at least one amyloid deposit in a subject suffering from a disease associated with amyloidosis.

24. Use of a compound according to formula (I): 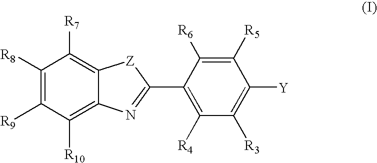
wherein
(i) Z is S, NR′, O or C(R′)₂, such that when Z is C(R′)₂, the tautomeric form of the heterocyclic ring may form an indole: 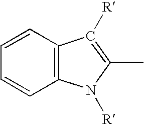
wherein R′ is H or a lower alkyl group,
(ii) Y is NR¹R², OR², or SR²,
(iii) R′ is selected from the group consisting of H, a lower alkyl group, (CH₂)ₙOR′ (wherein n=1, 2, or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3, or 4 and Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′ and COOR′, where R′ is H or a lower alkyl group);
(iv) R² is selected from the group consisting of H, a lower alkyl group, (CH₂)ₙOR′ (wherein n=1, 2, or 3), CF₃, CH₂—CH₂X, CH₂—CH₂—CH₂X (wherein X═F, Cl, Br or I), (C═O)—R′, Rₚₕ, and (CH₂)ₙRₚₕ (wherein n=1, 2, 3, or 4 and Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′ and COOR′, where R′ is H or a lower alkyl group);
(v) R³ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(vi) R⁴ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(vii) R⁵ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(viii) R⁶ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(ix) R⁷ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(x) R⁸ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(xi) R⁹ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
(xii) R¹⁰ is selected from the group consisting of H, F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, Rₚₕ, CR′═CR′—Rₚₕ, CR₂′—CR₂′—Rₚₕ (wherein Rₚₕ represents an unsubstituted or substituted phenyl group with the phenyl substituents being chosen from the group consisting of F, Cl, Br, I, a lower alkyl group, (CH₂)ₙOR′ (wherein n=1, 2, or 3), CF₃, CH₂—CH₂X, O—CH₂—CH₂X, CH₂—CH₂—CH₂X, O—CH₂—CH₂—CH₂X (wherein X═F, Cl, Br or I), CN, (C═O)—R′, N(R′)₂, NO₂, (C═O)N(R′)₂, O(CO)R′, OR′, SR′, COOR′, where R′ is H or a lower alkyl group) and a tri-alkyl tin;
alternatively, one of R³-R¹⁰ may be a chelating group, with or without a chelated metal group, said chelating group being of the form W-L or V-W-L, wherein V is selected from the group consisting of —COO—, —CO—, —CH₂O— and —CH₂NH—; W is —(CH₂)ₙ where n=0, 1, 2, 3, 4, or 5, and L is: 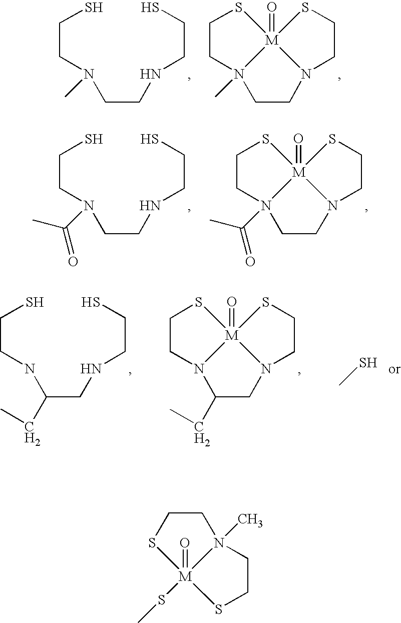
wherein M is selected from the group consisting of Tc and Re and radiolabelled derivatives and pharmaceutically acceptable salts thereof, where at least one of the substituent moieties comprises a detectable label,
in the preparation of a medicament for use in the detection of at least one amyloid deposit in a subject suffering from a disease associated with amyloidosis.
